(19) United States
(12) Patent Application Publication
(10) Pub. No.: US 2009/0087492 A1
(43) Pub. Date: Apr. 2, 2009

(54) Processes and Apparatuses for the Production of Crystalline Organic Microparticle Compositions by Micro-Milling and Crystallization on Micro-Seed and Their Use

(75) Inventors: Brian K. Johnson, Princeton, NJ (US); Hsien Hsin Tung, Edison, NJ (US); Ivan Lee, Piscataway, NJ (US); Aaron S. Cote, West Windosr, NJ (US); Cindy Starbuck, Branchburg, NJ (US); Michael Midler, Monroe Township, NJ (US)

Correspondence Address: PATENT DOCKET ADMINISTRATOR;LOWENSTEIN SANDLER PC, 65 LIVINGSTON AVENUE, ROSELAND, NJ, 07068, US

(73) Assignee: MERCK & CO., INC., Rahway, NJ (US)

(21) Appl. No.: 12/282,043

(22) PCT Filed: Mar. 12, 2007
(86) PCT No.: PCT/US2007/063785

§ 371 Date: Sep. 8, 2008

Related U.S. Application Data
(60) Provisional application No. 60/782,169, filed on Mar. 14, 2006.

(51) Int. Cl. A61K 9/14; C07F 9/02; A61K 31/66

(52) U.S. Cl. 424/489; 568/15; 514/75

(57) ABSTRACT

The present invention relates to a process, for the production of crystalline particles of an active organic compound The process includes the steps of generating a micro-seed by a wet-milling process and subjecting the micro-seed to a crystallization process. The resulting crystalline particles have a mean particle size of less than about 100 μm. The present invention also provides for a pharmaceutical composition which includes the crystalline particles produced by the method described herein and a pharmaceutically acceptable carrier.

### BACKGROUND OF THE INVENTION

During production of active organic compounds, such as, for example an active pharmaceutical ingredient (“API”), formation of solids is most often accomplished by crystallization in the solution phase followed by isolation and drying. Often times, the dry active organic compound must be further processed to reach a particle size profile necessary to ensure proper formulation of the end product. While, the resultant particle size can vary significantly, in most cases, fine pharmaceutical active ingredient powders have a mean size less than 300 um. However, there has been a strong need for crystals of a particle size less than 40 um due to pharmaceutical targets with low water solubility and/or low permeability. Small particles in a formulation provide higher surface area for transport into the body.

It is common to conduct a dry milling step, such as air jet classification milling, pin milling, or hammer milling to reach an acceptable particle size profile. Examples of dry milling equipment typically used for pharmaceutical processing include those produced by Hosakawa Micron (www.hosokawamicron.com) (eg. pin mill; Alpine® UPZ Fine Impact Mills, eg fluidized air jet mill: Alpine® AFG Fluidized Bed Opposed Jet Mills), those produced by Fluid Energy, those produced by Quadro Engineering and those described in Section 8 of Perry's Chemical Engineer's Handbook (Sixth edition ed. Robert H. Ferry and Don Green). The dry milling step can be used to either break agglomerates of particles into their native size and/or to break the native particles into smaller pieces.

From a process engineering point of view; dry milling introduces many operational concerns and costs. One major concern is the limitation of operator exposure to the active compounds. For highly potent compounds, dry milling may require expensive engineering controls to keep dusting low. Additionally, engineering controls may be necessary to minimize dust explosions. Other operational concerns of dry milling include accumulation of material inside the dry mill due to melting at high temperature or sticking to the internal components of the mill. In pin milling, this poor milling performance is commonly called “meltback” or “flagging,” respectively, and can even result in the production of amorphous material, mill plugging, and changes in the particle size exiting the mill as material is processed. Some compounds erode the mill during processing leading to unacceptable high levels of contaminants in the API product. Thus, it is desirable to form crystals of the target particle size distribution (PSD) directly from crystallization and avoid dry milling as the particle finishing step.

Unfortunately, methods of production directly via solution crystallization or directly via wet-milling techniques are lacking. One development is rotor-stator milling of a solid slurry followed by isolation. Rotor-stator milling typically produces particles of a mean size over 20 um. Unfortunately, in most cases, attrition is often seen in this milling process. Attrition occurs when very small particles are chipped off of the native particle leaving a bimodal particle size (American Pharmaceutical Review Vol 7, Issue 5, pp 120-123—“Rotor Stator Milling of API's . . . ”). Often times, rotor-stator milling results in a significantly slowed filtration step due to the presence of these fine particles. Additionally, formulation of bimodal feeds using direct compression or roller compaction techniques is problematic. The creation of a monomodal feed of small API particles would be beneficial in the absence of dry milling as a finishing step.

The formation of a new solid phase by crystallization, from solute dissolved in liquid, is generally accepted to occur by two pathways: (1) by nucleation of new particles or (2) by growth through deposition of solute on existing particles. Nucleation can occur on foreign substances in a crystallizer or homogeneously from solution. U.S. Pat. No. 5,314,506 entitled “Crystallization method to improve crystal structure and size” and U.S. Published Patent Application No. 2004/009.1546 A1 entitled “Process and apparatuses for preparing nanoparticle compositions with amphophilic copolymers and their use” describe small particles, even nanoparticles, produced by massive nucleation of many new particles of the solute during precipitation. In these processes, the character of the system is changed using solvent composition, temperature or reaction to create high supersaturation for the solute which in turn leads to rapid nucleation and crystallization. The birth of many particles by nucleation leads to a small particle size distribution at the end of the crystallization step, thereby obviating the need for dry milling.

A significant downside of the above nucleation processes is that under high supersaturation undesired solid state forms (crystal form/molecular packings in a crystal) can be produced as explained by Ostwald's rule (Threlfall—vol 7 no 6 2003 Organic Process Research and Development). The production of a variety of crystal forms was witnessed by Kabasci et al. for a calcium carbonate (Trans IChemE, vol 74, Part A, October 1996). If is common for pharmaceutical compounds to exhibit several different crystal forms for the same API and thus the use of these nucleation driven technologies are considered specialty applications. In addition, processes comprising high supersaturation and associated nucleation can yield crystals with occluded solvent molecules or impurities. In general, the purification and isolation process chosen for a pharmaceutical should yield a product of high chemical purity and the proper solid state form and processes dominated by nucleation events are not desirable.

In an effort to control the morphologic properties of the final product, it is a trend in fine particle engineering to use seed particles of the product to provide a template for crystal growth during crystallization. Seeding can help control the particle size, crystal form, and chemical purity by limiting the supersaturation. Various milling techniques have been employed to generate the seed stock. Dry milling has been used routinely to generate small particles for crystallization seed to result in particles of moderate size. This approach does not eliminate the previously discussed engineering and safety concerns associated with dry milling and is less desirable than a wet milling technique for seed generation.

It has been demonstrated that rotor-stator wet milling can be used to generate relatively large organic active particles with a practical limit of > 20 um. On the other hand, milling to >20 um requires extended milling time in the attrition regime where small fragments lead to a bimodal particle size distribution (American Pharmaceutical Review Vol 7, Issue 5, pp 120-123, “Rotor Stator Milling of API's . . . ). It has been found that crystallizations using rotor-stator wet milled products as seed result in large particles and, most often, a bimodal particle size distribution. A subsequent dry milling step is required to create tire desired small sized crystals or monomodal material. This method of seed generation is not ideal.

Sonication is another technique used to generate large seeds for crystallization. For example, sonication has been shown to yield product greater than 100 um (See U.S. Pat. No. 3,892,539 entitled “Process for production of crystals in fluidized bed crystallizers”). Media milling has recently been used to create final product streams for direct formulation of pharmaceuticals with particulates less than 400 mm (See U.S. Pat. No. 5,145,684), but using the wet milled micro-seed in a subsequent crystallization has not previously been shown. A review of media milling and its utilities is described in U.S. Pat. No. 6,634,576.

Tins patent describes possible materials for construction of the media mill and media mill beads. These include U.S. Pat. No. 3,804,653 which states that media can be formulated of sand, beads, cylinders, pellets, ceramic or plastic. This patent further discloses that the mill can be formulated of metal, steel alloy, ceramic and that the mill may be lined with ceramic. Plastic resin including polystyrene is noted as being particularly useful. U.S. Pat. No. 4,950,586 discloses the use of zirconium oxide beads to mill organic dyes to below 1 um in the presence of stabilizers. Several combinations of mill construction may be used to practice the instant invention. In one embodiment, ceramic beads and a ceramic mill are utilized. In a further embodiment, ceramic beads and a chromium-lined mill are utilized.

In summary, there remains a need for crystallization processes that can produce organic actives and especially pharmaceutical products at a controlled size or surface area sufficient to obviate dry milling to meet formulation demands. The pharmaceutical industry is consistently requiring smaller particles due to their increased bioavailability and/or dissolution rate. Likewise, it is also important to yield chemical compounds with the requisite crystal form and a well-controlled crystal purity. In the present invention, wet milled micro-seed with a mean particle size ranging from about 0.1 to about 20 um has been shown to be surprisingly effective for the production of fine organic active solid particles, and especially for the crystallization of active pharmaceuticals ingredients, with a controlled particle size distribution, crystal form, and purity. Further advantages of the present invention include the elimination of the need for downstream milling, thereby eliminating the health and safety hazards often associated with these processes.

### SUMMARY OF THE INVENTION

The present invention provides a process for the production of crystalline particles of an organic active compound. The process includes the steps of generating a micro-seed by a wet-milling process and subjecting the micro-seed to a crystallization process. The micro-seed generated by the wet milling process has a mean particle size of about 0.1 to about 20 μm. The resulting crystalline particles have a mean particle size of less than 100 μm.

With respect to the crystallisation step, the present invention includes two methods. The first crystallization method is a three-step process: generating a slurry of the micro seed using media milling; dissolving a portion of the micro-seed; and crystallizing the active organic compound on the micro-seed.

The second crystallization method is also a three-step process including generating a slurry of the micro-seed; generating a solution of the product to be crystallized; and combining the slurry with the solution. In one embodiment of this second crystallization process, the slurry of the micro-seed and the solution of the product are rapidly micro-mixed when they are combined.

One of three processing configurations may be used individually or in combination in order to accomplish the second crystallization method. One configuration is a batch processing; another is a semi-continuous processing; a third is a continuous processing configuration.

A recycle loop may also be used in conjunction with the second crystallization process. In one embodiment of the second crystallization process, a recycle loop is utilized as part of the batch processing configuration. In another embodiment of the second crystallization process, a recycle loop is utilized as part of the semi-continuous processing configuration. In yet another embodiment of the second crystallization process, a recycle loop is utilized as part of the continuous processing configuration.

The second crystallization method uses two types of solvent streams. In one embodiment, the solvent system is an aqueous solvent stream; in another, the solvent system is an organic solvent stream; in yet another, the solvent system is a mixed solvent stream.

Additionally, a supplemental energy device may be used in conjunction with the second crystallization process, in a first embodiment, this supplemental energy device is a mixing tee; in a second, it is a mixing elbow; in a third it is a static mixer; in a fourth, it is a sonicator; and, in a fifth, it is a rotor-stator homogenizer.

Further, the active organic compound of the present invention may be a pharmaceutical selected from a group which includes analgesics, antiinflammatory agents, anthelmintics, anti-arrthymics, anti-asthmatics, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytics, sedatives, astringents, beta-adrenergic receptor blocking drugs, contrast media, corticosteroids, cough suppressants, diagnostic agents, diagnostic imaging agents, dopaminergics, haemostatics, immunological agents, lipid regulating agents, muscle relaxants, parasympathomimetics, parathyroid calcitonin, prostaglandins, radio-pharmaceuticals, sex hormones, anti-allergic agents, stimulants, sympathomimetics, thyroid agents, vasodilators and xanthines.

Additionally, the present invention further provides a pharmaceutical composition including the crystalline particles produced by the processes described herein and a pharmaceutically acceptable carrier.

### BRIEF DESCRIPTION OF THE FIGURES

FIG. 1 demonstrates the typical components necessary for media milling in recycle mode, including the blending vessel, fluid pump, media mill, and recycle line back to the vessel. Single pass milling does not recycle and Simply feeds the product into a collection receiver through the mill. In single pass mode, the pump can be replaced by a pressure transfer from the still. Multiple single passes can accomplish a similar product profile as the recycle mode.

FIG. 2 demonstrates a crystallization vessel set up for Examples 1-7 and 9. In Example 1, the antisolvent was charged rapidly < 10 seconds in portions using a syringe with a needle. Optionally, a sonicator probe and or a light scattering probe can be added.

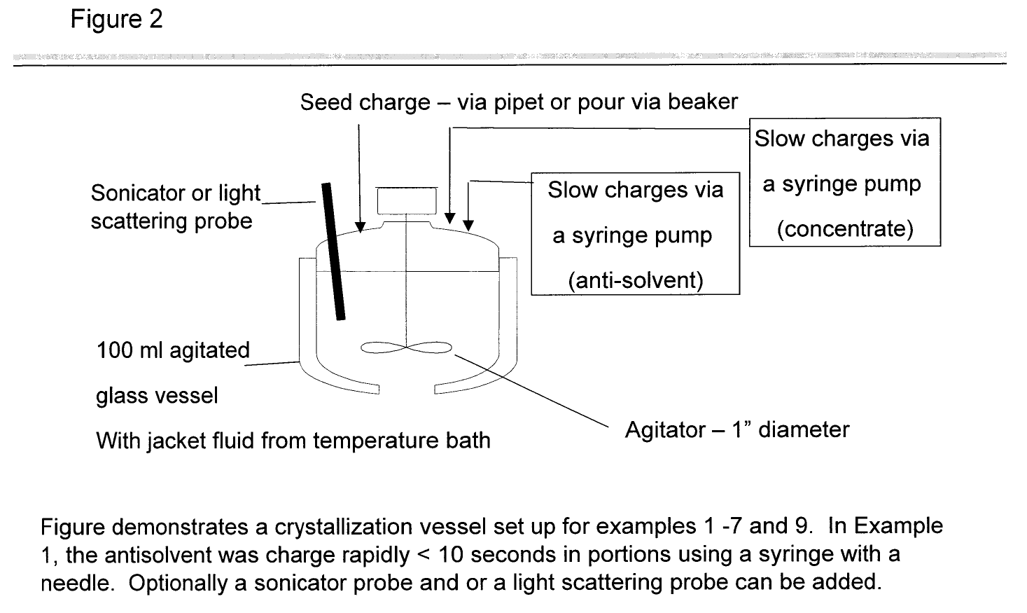

FIG. 3 displays an example set-up which was shown amenable for scale up of the micro-milling and crystallization process as in Example 10, 11, and 12. The crystallization vessel and components of the recycle loop are presented.

FIG. 4 displays the process discussed in Example 8, wherein an external recycle loop is employed for the application of a supplemental energy device. The energy devices are motionless where the fluid flow through the mixer provides energy input into the system by pressure drop aid turbulent fluid movement. The double tee consisted of two tees arranged as pictured which promotes the impingement of two streams and the static mixer was that of the “kenics helical style” manufactured by Koflo Corp.

FIG. 5 demonstrates the double tee supplemental energy device used in Example 11. The lines are made of ¾″ ID steel pipe with sharp right angle turns. The streams impinge at the outlet.

FIG. 6 is a general overview of a possible crystallization process, including generating a slurry of the micro-seed; generating a concentrate solution of the product to be crystallized; and combining the slurry with the concentrate to imitate crystallization. Further crystallization can be afforded by a number of methods to create supersaturation, some of which are listed.

FIG. 7 is an example of a batch crystallization method.

FIG. 8 is an example of a semi-continuous crystallization method.

FIG. 9 is an example of a batch reactive crystallization method. Shown is a reaction scenario where reagent A and B react to form the product to be crystallized.

FIG. 10 is a micrograph of the product of Example 1B.

FIG. 11 is a micrograph of the product in the micro-milling process for Example 3B after 0.5 minutes of recycle micro-milling.

FIG. 12 is a micrograph of the product in the micro-milling process for Example 3B after 15 minutes of recycle micro-milling.

FIG. 13 is a micrograph of the product in the micro-milling process for Example 3B after 60 minutes of recycle micro-milling.

FIG. 14 is a micrograph of the product slurry at the end of crystallization of Example 3B.

FIG. 15 is a micrograph of the product slurry at the end of crystallization of Example 4B.

FIG. 16 is a micrograph of the product slurry at the end of crystallization of Example 5.

FIG. 17 is a micrograph of the product slurry at the end of crystallization of Example 8A.

FIG. 18 is a micrograph of the product slurry at the end of crystallization of Example 8B.

FIG. 19 is a micrograph of the product slurry at the end of crystallization of Example 9A.

FIG. 20 is a micrograph of the product slum at the end of crystallization of Example 9B.

FIG. 21 is a micrograph of the product slurry at the end of crystallization of Example 10.

FIG. 22 is a micrograph of the product slum at the end of crystallization of Example 11.

FIG. 23 is a micrograph of the product slurry at the end of crystallization of Example 12.

FIG. 24 is a particle size distribution report for the product in the micro-milling process for Example 3B after 15 minutes of recycle micro-milling.

FIG. 25 is a particle size distribution report for the product in the micro-milling process for Example 3B after 60 minutes of recycle micro-milling.

FIG. 26 is a report on the pharmacokinetic data collected for three dogs comparing the plasma level of compound F in the bloodstream for the first 24 hours after ingestion of a direct fill capsule for the micro-milling and crystallization process or dry milling process as in Example 6.

### DETAILED DESCRIPTION OF THE INVENTION

The micro-milling and crystallization process (“MMC”) of the present invention comprises growth on micro-seed particles to a mean volume particle size less than about 100 um, such as for example, less than about 60 um, further still less than about 40 um. In most cases the product will range from about 3 to about 40 um depending on the amount of seed added for crystallization. The micro-seed can range from about 0.1 to about 20 um, for example, from about 1 to about 10 um by mean volume analysis. The seed can be generated by a number of wet milling devices, such as for example, media milling. Particles less than 1 um mean may also be utilized. However, this size range is less attractive than micro-seed because the resulting API particle sizes if the particles are kept dispersed during a growth crystallization are smaller titan desired for conventional isolation techniques using typical seed levels of about 0.5% to about 15%.

The process of the present invention (MMC) comprises generating a slurry of the micro-seed and generating a solution containing the product to be crystallized. These two streams are combined to provide crystallization of the product. In most cases, the crystallization is continued by manipulating changes in product solubility and concentration in order to drive the crystallization. These manipulations lead to a supersaturated system which provides a driving force for the deposition of solute on the seed. The level of supersaturation during the seeding event and the subsequent crystallization is controlled at a level to enhance growth conditions versus nucleation. In the present invention, the process is designed to facilitate growth on the micro-seed while controlling the birth of new particles. A review of the methods for crystallization including a discussion of growth and nucleation process conditions is provided by Price (Chemical Engineering Progress, September 1997, P34 “Take some Solid Steps to improve Crystallization”).

The micro-seed and product particles of the MMC process of the present invention have a number of specific advantages. The micro-seed particles have a high surface area to volume ratio and thus the growth rate, at a given supersaturation, is enhanced significantly relative to large seed particles. A high population of seed particles avoids nucleation on foreign substances and the crystallization is one of growth on the existing seed particles at low supersaturation. Thus, the size and form of the API particles are controlled by the characteristics of the seed particle.

Generally, operating at reactor conditions where the desired crystal form is the most stable and seeding with the desired crystal form is preferred. It has been discovered that small particles have less sensitivity to particle attrition by shear since the particle-particle impacts are between objects of significantly less weight. Starting with monomodal seed, the process of the present invention provides a monomodal particle size distribution as confirmed by optical micrographs and laser scattering techniques. Due to the monodisperse particle size of the resultant product, it is amenable to downstream filtration and formulation making the composite process an attractive method for fine particle finishing.

Although the present invention may be utilized for the production of any precipitated or crystallized organic active particles, including pharmaceuticals, biopharmaceuticals, nutraceuticals, diagnostic agents, agrochemicals, insecticides, herbicides, pigments, food ingredients, food formulations, beverages, fine chemicals, and cosmetics; for ease of description, principally pharmaceuticals will be specifically addressed. The crystalline/precipitated particles for organic compounds used in other industry segments can be produced using the same general techniques described herein.

Any method of generating a supersaturation to promote growth in the presence of the micro-seed is amenable to this invention. Common methods to manipulate crystallization include changes in solvent composition, temperature, use of chemical reaction, or use of distillation. Although reactive crystallization requires the formation of the final API from one or more reagents, the APT formed becomes supersaturated and supersaturation of the product is the source of crystallization. A review of crystallisation methods to generate supersaturation and the interplay between nucleation and growth is provided by Price (Chemical Engineering Progress, September 1997, P 34 “Take some Solid Steps to Improve Crystallization”). This reference, in its entirety, is hereby incorporated by reference into the subject application.

The addition of the micro-seed to the solute or the solute to the micro-seed can be accomplished in several ways including batch crystallization, semi-batch crystallization or semi-continuous crystallization. These techniques are common to those practiced in the art and extensions to other crystallizer configurations are expected. Additionally, a combination of these methods can be utilized.

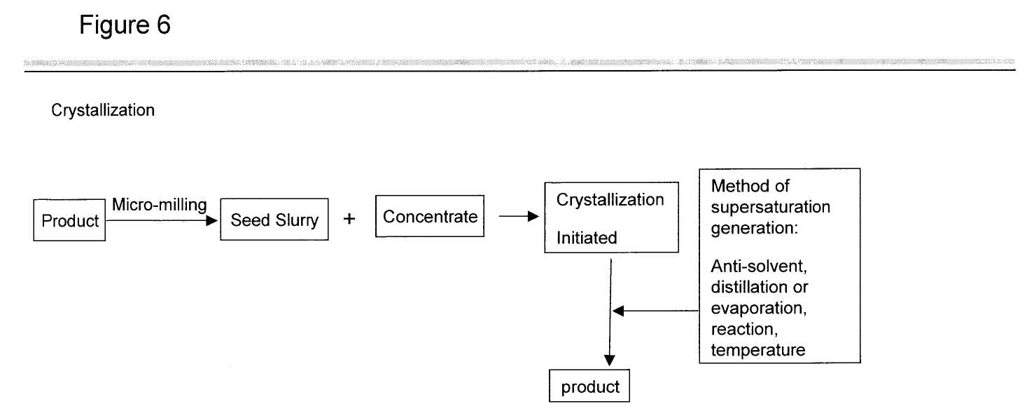

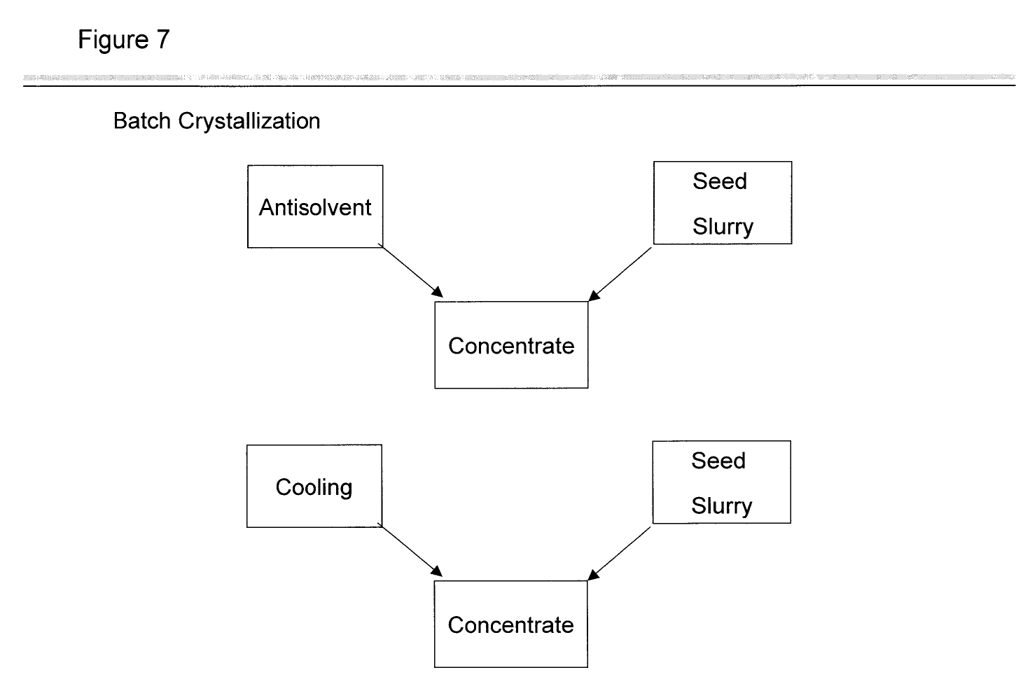

Batch crystallization typically includes crystallizations where the temperature is changed or solvent is removed by distillation to generate the supersaturation. A semi-batch crystallization typically includes the continuous addition of a solvent or reagent to a reservoir of solute or the reaction precursor for the solute. In batch and semi-batch crystallization, the seed is typically added to a reservoir of solute which is supersaturated at the time of seed addition or as a result of the seed addition. See FIGS. 6 and 7.

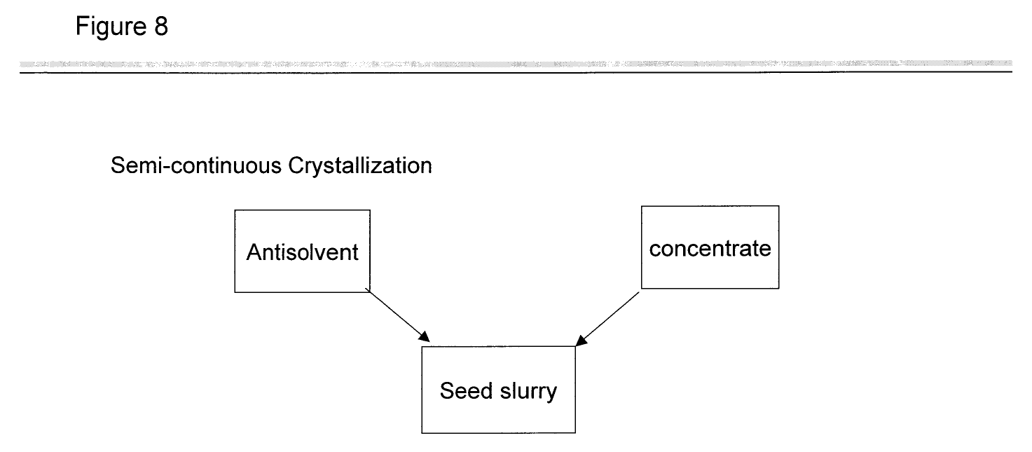

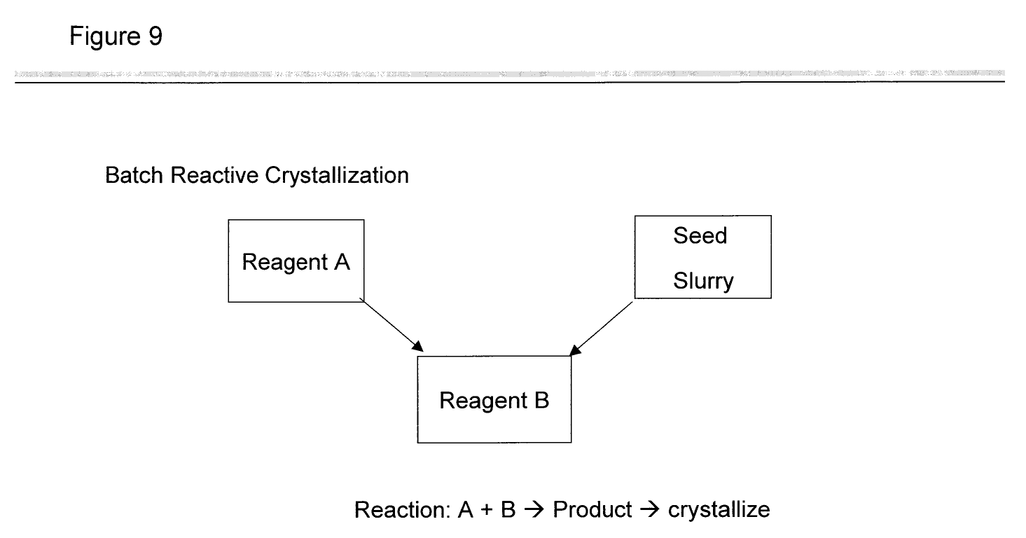

Semi-continuous crystallization is designed to keep the contents of the liquid phase in the reactor nearly constant throughout the crystallization. In a semicrontinuous crystallization by non-solvent (also called an anti-solvent), a seed stream is added to a reactor followed by the simultaneous addition of both a stream containing the solute dissolved in solution and a stream of non solvent. Here the crystallization occurs at a rate similar to the rate at which the components are added. See FIG. 8. An example schematic for a reactive crystallization is provided in FIG. 9.

The chemical composition of the streams chosen for the MMC process is dependent on the compound being crystallized. Accordingly, aqueous, organic or mixed aqueous and organic streams can be utilized.

In the process of the present invention, wet milling to micro-seed size is required to limit the need for dry-milling in a downstream production process. Only select machines can provide particles of a mean optimum size ranging from about 1 to about 10 um. Milling methods such as high energy hydrodynamic cavitation or high intensity sonication, high energy ball or media milling, and high pressure homogenization are representative of the technologies that can be utilized to make micro-seed having a mean optimum size ranging from about 1 to about 10 um.

In one embodiment of the invention media milling is an effective wet milling method to reduce the particle size of seed to the target size. In addition, media milling has been found to maintain the crystallinity of the API upon the milling process. The size of the media beads utilized ranges, for example, from about 0.5 to about 4 mm.

Additional parameters that cart be changed during the wet milling process of the invention, include product concentration, milling temperature, and mill speed to afford the desired micro-seed size.

Media milling work on API product streams has been practiced to generate particles less than one micron in mean size using specialty beads of 0.5 mm or less in the presence of colloidal stabilizers. The surface active agents overcome the colloidal forces that are active at less than one micron aid provide a stream of disperse particles for formulation. This feed stream can be used in the current invention as micro seed. Crystallizations from the current invention are most predictable when a substantially disperse seed is utilized for crystallization. Using aggregates of particles as seed is less desirable since the number and Size of the aggregates could be variable. Thus, seed crystals of 0.1 um to 0.5 um may be utilized in the present invention where it is desirable to employ colloidal stabilizers unless the organic compound is self-stabilized as disperse particles.

Since the process of the present invention is primarily one of growth on existing seed particles, the amount and size of micro-seed is the primary determinant of the API particle size. Variable amounts of seed can be added to afford the desired particle size distribution (PSD) after crystallization. Typical seed amounts (material not dissolved in the solvent phase of the seed slurry) range from about 0.1 to 20 wt % relative to the amount of the active ingredient to be crystallized. In a growth crystallization, introduction of less seed leads to larger particles. For example, low amounts of seed can increase the product particles size above 60 um, but the crystallization could potentially be very slow to avoid nucleation and promote growth on those seeds. Seed levels of about 0.5 to 15% are reasonable charges starting with micro-seed of 1 to 10 um.

In another embodiment, the MMC process comprises
(1) using a wet milling process to generate micro-seed having a mean size of approximately 0.1 to 20 μm; and
(2) crystallizing an organic active compound on the micro-seed to yield crystalline particles having a mean size less than 100 μm.

In a further embodiment the MMC process comprises:
(1) using a wet milling process to generate micro-seed having a mean size of approximately 0.1 to 20 μm;
(2) dissolving a portion of the micro-seed; and
(3) crystallizing an organic active compound on the micro-seed to yield crystalline particles having a mean size less than 100 μm.

The dissolution process may comprise heating, changes in pH, changes in solvent composition or other. This tailors the resultant particle size distribution to one only slightly larger than the seed. In some cases only mild enhancement of the micro-seed particle size is sufficient for the product needs and thus seed levels of 50% or higher may be used.

In one embodiment the micro-seed may be isolated and charged as a dry product.

The MMC process of the current invention is highly scalable. Proper equipment design at each scale may enable robust performance at all scales. Two features that may be employed for reliable scale up: 1) rapid micro-mixing during additions of materials to an actively crystallizing system and 2) inclusion of an energy device for particle dispersion of unwanted agglomeration. Crystallizer designs containing these features are amenable for scale-up of the invention.

Rapid micro-mixing implies a fast mixing time of the two streams at the molecular level relative to the characteristic induction time for crystallization of the product. These concepts are explained in detail by Johnson and Prud'homme (Australian Journal of Chemistry 56(10): 1021-1024 (2003)) and by Marcant and David (AIChE Journal November 1991 vol 37. No 11). Both groups of authors stress that the micro-mixing time can affect the outcome of a crystallization or precipitation. Accordingly, the authors emphasize that a low micro-mixing time is advantageous. For solvent, concentrate, or reagent additions, this rapid micro-mixing reduces or eliminates concentration gradients that could lead to a nucleation event.

In one embodiment of the invention, supersaturation is kept low to promote growth on the micro-seed. In some cases, the kinetics of crystallization are fast and nucleation cannot be substantially avoided. An appropriate rapid mixer should be chosen in these cases to limit nucleation by mixing reagent streams quickly and avoiding high local concentrations of reagents. When the micro-seed is added to a crystallizer containing solute, dispersion of the seed by rapid micro-mixing is important to limit agglomeration of the micro-seed as crystallization takes place.

Additionally, the work of Hunslow (Chemical Engineering Transactions, “Proceedings of the 15^{th} International Symposium on Industrial Crystallization 2002”; Volume 1 2002, p 65, published by ADIC—Associazione Italiana Di Engegneria Chemi) teaches that agglomeration of particles is directly related to the level of local supersaturation. Therefore, rapid micro-mixing is also helpful in minimizing agglomeration for this situation. The selection of a rapid mixer must be balanced against the level of particle attrition by the choice of the mixer. The mechanism leading to particle birth due to particle-particles or particles-crystallizer surface interactions in the presence of seed particles is commonly referred to as secondary nucleation and is expected to occur to some extent in most crystallizations. The choices of equipment can alter the extent of this effect.

Organic active compounds of small size have a tendency to aggregate and then agglomerate by the deposition of mass on an aggregate during crystallization. When particles agglomerate the API particle size will be larger than if growth occurred only on the individual seed particles and agglomerates were not present. In some pharmaceutical applications, agglomeration is not desired for it can be more difficult to scale up a process comprising agglomerated particles, in these situations, it is desirable to develop methods to use the micro-seed where agglomeration is managed.

In general, the energy density experienced by the particles must be sufficient to afford deagglomeration and the particles must be exposed to the energy density during crystallization at a frequency sufficient to maintain a disperse system. A supplemental energy device helps to minimize agglomeration by dispersing particles. A function of the energy device is to create particle collisions which break lightly agglomerated materials apart or create a shear filed which torque and break the agglomerates. This energy device could be as simple as a properly designed tank agitator or a recycle pipe with fluid pumping through it. Fluid pumps are high energy devices and can affect the crystallization process. These devices are sufficient when aggregates and agglomerates are not strong or the product is exposed to the device frequently. Rotor stator wet-mills are useful to provide a strong shear environment and are most useful when the particles themselves are not attritted. Sonication energy applied to the crystallizer has been found to limit agglomeration of compounds that aggregate readily and form stronger agglomerates. Applying sonication or an energy device at the end of the crystallization can also be useful to break agglomerates, but is less desirable than during the crystallization since the agglomerates may be of significant strength by the end of the crystallization time. Sonication horns also provide a sound wave which may be responsible for breaking lightly agglomerated materials without fracturing the primary particles.

Needle crystals present challenges for the processing of fine organics. In particular, their filtration rates are typically slow. One aspect of this invention is the use of sonication during crystallization. Sonication can promote the growth of needle crystals in the width direction yielding a more robust product for filtration. The use of sonication to generate micro-seed for needle crystals is also especially advantageous. Needles tend to break on the long axis and produce crystals of a similar width, but shorter length.

The fundamental technology of sonication (ultrasound waves typically between 10 and 60 kHz) is highly complex and the fundamental mechanism for successful deagglomeration is unclear, hut it is well known that sonication is effective at deaggregation or deagglomeration (Pohl and Schubert Partec 2004 “dispersion and deagglomeration of nanoparticles in aqueous solutions”). As a nonbinding explanation of the mechanical process, sonication provides ultrasound waves of a high power density and thus a high strength for agglomerate disruption. Cavitation bubbles are formed during the negative-pressure period of the wave and the rapid collapse of these bubbles provide a shock wave and high temperature and pressures useful for deagglomeration. In the present invention, it has been found that the seed and grown particles are not significantly fractured in most cases, and thus, the high energy events of sonication are especially effective to promote growth on disperse particles without attrition of the particles.

In the recent years, work on sonication for chemistry has strayed into crystallization. Focus has been placed on the use of ultrasound to reduce the induction time for nucleation or to provide facile nucleation at moderate supersaturation. This is useful to enhance the reproducibility of seed bed generation in the absence of solids apriori or without needing to add a solid seed to the batch concentrate (McCausland et. al. Chemical Engineering Progress July 2001 P 56-61). This approach is contrary to the current teachings where the presence of micro-seed dictates the final product properties and especially the crystal form.

The application of sonication to pharmaceutical crystallisation for the purpose of controlled growth on disperse micro-seed particles as in the MMC process is unique. In addition, the sonication power required for successful deagglomeration as demonstrated in the current invention is relatively small, less than 10 watts per liter of total batch at the end of crystallization and preferably less than 1 watt per liter of total batch at the end of crystallization. The design of equipment for sonication and research into the technology is an active area of research. Examples of flow cells amenable to the present invention are commercially provided by several manufactures (eg. Branson WF3-16) and (eg. Telsonics SRR46 series) for use in recycle loops as an energy device.

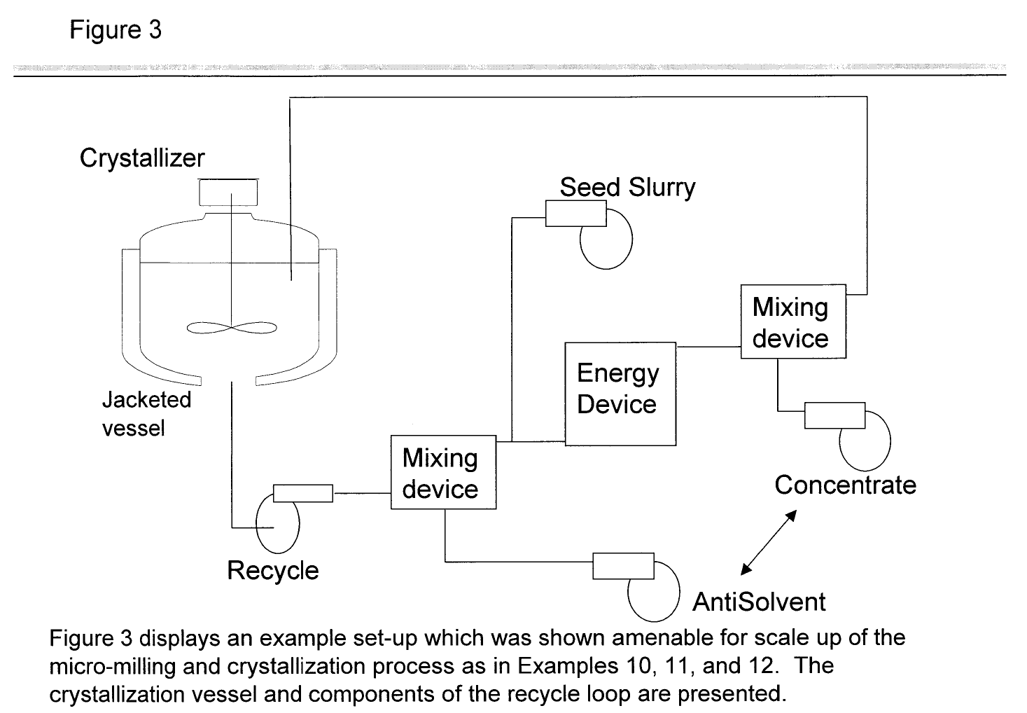

The use of a recycle loop to provide methods for micro-mixing and methods to incorporate a supplemental energy device has been shown to be especially advantageous for scale up. The primary concept is to relieve the micro-mixing and energy input demands from a conventional crystallizer (typically a stirred tank) and create specialized zones of functionality. The stirred tank crystallizer can serve as a blending device, with micro-mixing and supplemental energy input to the system independently controlled external to the tank. This approach is an example of a scalable crystallization system for large scale production. A practical emulation of tins system is provided in FIG. 3. Micro-mixing is best accomplished by adding a stream into a region of high energy dissipation or high turbulence. Addition of the stream into the center of the pipe into a region of turbulent flow in a recycle loop is one embodiment. In this case, a velocity of at least 1 m/s is recommended for conventional pipe flow, but not essential provided the micro-mixing is fast. This example is not limiting for the location of reagent addition and method of reagent addition is critical to achieving proper micromixing. The concepts of mixing in pipelines and in stirred vessels are described in The Handbook of Industrial Mixing (Ed. Paul et al, 2004, Wiley Interscience).

The recycle rate for the crystallizer can be quantified by the time to pass the equivalent of one volume of the batch at the end of the crystallization through the recycle loop, or the turnover time at the end of the crystallization. The turnover time for a vessel can be varied independently and will be a function of the frequency at which the batch should be exposed to the supplemental energy device to limit the agglomeration of the product. A typical turnover time for large scale production ranges from about 5 to about 30 minutes, but this is not limiting. Since the agglomeration of the product crystals typically requires deposition of mass by crystallization, the rate of crystallization can be slowed to extend the turnover time required to afford deagglomeration.

The particle size and surface area of the resultant product may be enhanced by the addition of supplemental additives to the seed or the crystallization batch, in one embodiment, the additives help disperse the seed and crystals in the crystallizer which limits particle agglomeration. The addition of supplemental additives may be used for other purposes as well, such as reduction of product oxidation or to limit compounds sticking to the sides of a vessel. The supplemental additives may be substantially removed by the isolation step yielding a pure active ingredient. Materials with surfactant properties are useful to enhance the slurry characteristics of the milling, seeding, and crystallization steps of the MMC process.

Supplemental additives include, but are not limited to: inert diluents, amphophilic copolymers, solubilizing agents, emulsifiers, suspending agents, adjuvants, wetting agents, sweetening, flavoring, and perfuming agents, isotonic agents, colloidal dispersants and surfactants such as but not limited to a charged phospholipid such as dimyristoyl phophatidyl glycerol; alginic acid, alignates, acacia, gum acacia. 1,3 butyleneglycol, benzalkonium chloride, collodial silicon dioxide, cetostearyl alcohol, cetomacrogol emulsifying wax, casein, calcium stearate, cetyl pyridinium chloride, cetyl alcohol, cholesterol, calcium carbonate, Crodestas F-110®, which is a mixture of sucrose stearate and sucrose distearate (of Croda Inc.), clays, kaolin and bentonite, derivatives of cellulose and their salts such as hydroxy propyl methylcellulose (HPMC), carboxymethylcellose sodium, carboxymethylcellulose and its salts, hydroxy propyl celluloses, methyl cellulose, hydroxyethylcellulose, hydroxypropylcellulose, hydroxypropylmethylcellulose phthalate, noncrystalline cellulose; dicalcium phosphate, dodecyl trimethyl ammonium bromide, dextran, dialkylesters of sodium sulfosuccinic (e.g. Aerosol OT® of American Cyanamid), gelatin, glycerol, glycerol monostearate, glucose, p-isononylphenoxypolt-(glycidol), also known as Olin 10-G® or surfactant 10-G® (of Olin Chemicals, Stamford, Conn.); glucamides such as octanoyl-N-methylglucamide, decanoyl-N-methylglucamide; heptanoyl-M-methylglucamide, lactose, lecithin(phosphatides), maltosides such as n-dodecyl β-D-maltoside; mannitol, magnesium stearate, magnesium aluminum silicate, oils such as cotton seed oil, corn germ oil, olive oil, castor oil, and sesame oil; paraffin, potato starch, polyethylene glycols (eg the Carbowaxs 3350® and 3450®, and Carbopol 934® of Union Carbide), polyoxy ethylene alkyl ethers (eg. macrogol ethers such as cetomacrogol 1000), polyoxy ethylene sorbitan fatty acid esters (eg. the commercially available Tweens® of ICI specialty chemicals), polyoxy ethylene castor oil derivatives, polyoxyethylene sterates, polyvinylalcohol (PVA), polyvinylpyrrolidone (PVP), phosphates, 4-(1,1,3,3-tetramethylbutyl)phenol polymer with ethylene oxide and formaldehyde, (also known as tyloxapol, superione, and triton), all poloxamers and polaxamines (e.g. Pluronics F68LF®, F87®, F108® and tetronic 908® available from BASF Corporation Mount Olive, N.J.), pyranosides such as n-hexyl β-D-glucopyranoside, n-heptyl β-D-glucopyranoside; n-octyl-β-D-glucopyranoside, n-decyl β-D-glucopyranoside; n-decyl β-D-maltopyranoside: n-dodecyl β-D-glucopyranoside; quaternary ammonium compounds, silicic acid, sodium citrate, starches, sorbitan esters, sodium carbonate, solid polyethylene glycols, sodium dodecyl sulfate, sodium lauryl sulfate (eg. DUPONOL P® of DuPont corporation), steric acid, sucrose, tapioca starch, talc, rhioglucosides such as n-heptyl β-D-thioglucoside, tragacanth, triethanolamine, Triton X-200® which is a alkyl aryl poly ether sulfonate (of Rhom and Haas); and the like. The inert diluents, solubilizing agents, emulsifiers, adjuvants, wetting agents, isotonic agents, colloidal dispersants and surfactants are commercially available or can be prepared by techniques known in the art.

Likewise it is possible to synthesize desirable chemical structures not commercially available, such as crystal growth modifiers to tailor the process performance. The properties of many of these and other pharmaceutical excipients suitable for addition to the process solvent streams before or after mixing are provided in the Handbook of Pharmaceutical Excipients, 3rd edition, editor Arthur H. Kibbe, 2000, American Pharmaceutical Association, London, the disclosure of which is hereby incorporated by reference in its entirety.

in the MMC process of the present invention, microparticles are formed in the final mixed solution. The final solvent concentration containing the microparticles can be altered by a number of post treatment processes, including, but not limited to, dialysis, distillation, wiped film evaporation, centrifugation, lyophilization, filtration, sterile filtration, extraction, supercritical fluid extraction, and spray drying. These processes typically occur after formation of the microparticles, but could also occur during the formation process.

It has been noted that a high solubility of product in the solution phase can during drying lead to deposition of residual solute in the liquid phase on the particles leading to light agglomerates of the native particles formed during crystallization. Dissolution of a drug particle after formulation is often sensitive to the surface area of the native particle size versus agglomerates. The Sight agglomerates can be broken during formulation processing to yield products with acceptable bioavailability.

In measuring particle size, care must be taken to select the correct measuring tool. For instance, typical laser light scattering techniques used to measure particle size may result in erroneous readings since the techniques employed may not be able to break agglomerates into native particles. Thus, particle size analysis of the product may indicate large agglomerates instead of the native particle size. Measurement of the surface area versus light scattering techniques is a preferred measurement technique as set form in the examples below. However, mean particle size may also be measured using conventional laser light scattering devices. Specifically, the analysis of dry product is preferred in a machine similar to the Sympatec Helos machine with 1 to 3 atm pressure. In general, the surface area of a product and the particle size are directly related depending on the shape of the particle in question.

One shape of a particle that is often problematic for particle size analysis is that of needles where the aspect, ratio of the length to width is greater than 6. This type of a particle can demonstrate a hi modal particle size distribution when micrographs show a consistent product of small size has been produced. For this invention, the particle size by light scattering in dry analysis cell is measured in a Sympatec Helos when the aspect ratio is less than 6. When the aspect ratio is 6 or greater, optical microscopy is used to measure the particle size by the longest dimension of the crystal.

Subsequent post processing of the product of a MMC process into a pharmaceutical formulation is typically advantageous to enhance the product performance or product acceptance as a marketed product. Processes such as, but not limited to, roller compaction, wet granulation, direct compression, or direct fill capsules are all possible. In particular, pharmaceutical compositions with the product of the MMC process can be made to satisfy the needs of the industry and these formulations include supplemental additives of various types as stated above. Possible but not limiting classes of compounds for the MMC process and subsequent formulation include: analgesics, anti-inflammatory agents, antihelmintics, anti-arrthymics, anti-asthmatics, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytics, sedatives, astringents, beta-adrenergic receptor blocking drugs, contrast media, corticosteroids, cough suppressants, diagnostic agents, diagnostic imaging agents, dopaminergics, haemostatics, immunological agents, lipid regulating agents, muscle relaxants, parasympathomimetics, parathyroid calcitonin, prostaglandins, radio-pharmaceuticals, sex hormones, anti-allergic agents, stimulants, sympathomimetics, thyroid agents, vasodilators and xanthines. Drug substances include those intended for oral administration and intravenous administration and inhalation administration although it is conceivable to use other methods such as dermal patches. The drug substances can be selected from any pharmaceutical organic active and precursor compound. A description of these classes of drugs and a listing of species within each class can be found in Physicians Desk Reference. 51 edition, 2001, Medical Economics Co., Montvale, N.J., the disclosure of which is hereby incorporated by reference in its entirety. The drug substances are commercially available and/or can be prepared by techniques known in the art.

As used herein, the terms “crystallization” and/or “precipitation” include any methodology of producing particles from fluids: including, but not limited to classical solvent/antisolvent crystallization/precipitation; temperature dependent crystallization/precipitation; “salting out” crystallization/precipitation; pH dependent reactions; “cooling driven” crystallization/precipitation; crystallization/precipitation based upon chemical and/or physical reactions, etc.

As used herein, the term “biopharmaceutical” includes any therapeutic compound being derived from a biological source or chemically synthesized to be equivalent to a product from a biological source, for example, a protein, a peptide, a vaccine, a nucleic acid, an immunoglobulin, a polysaccharide, cell product, a plant extract, an animal extract, a recombinant protein an enzyme or combinations thereof.

As used herein, the terms “solvent” and “anti-solvent” denote, respectively, those fluids in which a substance is substantially dissolved, and a fluid which causes the desired substance to crystallize/precipitate or fall out of solution.

The process and apparatus of the present invention can be utilized to crystallize a wide variety of pharmaceutical substances. The water soluble and water insoluble pharmaceutical substances that can be crystallized according to the present invention include, but are not limited to, anabolic steroids, analeptics, analgesics, anesthetics, antacids, anti-arrthymics, anti-asthmatics, antibiotics, anti-cariogenics, anticoagulants, anticolonergics, anticonvulsants, antidepressants, antidiabetics, antidiarrheals, anti-emetics, anti-epileptics, antifungals, antihelmintics, antihemorrhoidals, antihistamines, antihormones, antihypertensives, antihypotensives, anti-inflammatories, antimuscarinics, antimycotics, antineoplastics, anti-obesity drugs, antiplaque agents, antiprotozoals, antipsychotics, antiseptics, anti-spasmotics, anti-thrombics, antitussives, antivirals, anxiolytics, astringensts, beta-adrenergic receptor blocking drugs, bile acids, breath fresheners, bronchospasmolytic drugs, bronchodilators, calcium channel blockers, cardiac glycosides, contraceptives, corticosteroids, decongestants, diagnostics, digestives, diuretics, dopaminergics, electrolytes, emetics, expectorants, haemostatic drugs, hormones, hormone replacement therapy drugs, hypnotics, hypoglycemic drugs, immunosuppressants, impotence drugs, laxatives, lipid regulators, mucolytics, muscle relaxants, non-steroidal anti-inflammatories, nutraceuticals, pain relievers, parasympatholytics, parasympathomimetics, prostagladins, psychostimulants, psychotropics, sedatives, sex steroids, spasmolytics, steroids, stimulants, sulfonamides, sympatholytics, sumpathicomimetics, sympathomimetics, thyreomimetics, thyreostatic drugs, vasodilators, vitamins, xanthines and mixtures thereof.

Pharmaceutical compositions according to this invention include the particles described herein and a pharmaceutically acceptable carrier. Suitable pharmaceutically acceptable carriers are well known to those skilled in the art. These include non-toxic physiologically acceptable carriers, adjuvants or vehicles for parenteral injection, for oral administration in solid or liquid form, for rectal administration, and the like. The pharmaceutical compositions of this invention are useful in oral and parenteral, including intravenous, administration applications but this is not limiting.

The following examples provide a non limiting description of methods to exercise the MMC process of the present invention.

For the foil owing examples:

Micro-seed particles were made by one of two mills: The 600 ml disc mill represented a KDL model made by DYNO®-Mill. The mill chamber was chromium treated and the agitating discs were yttrium stabilized zirconium oxide. The mill was charged with approximately 1900 grams of yttrium stabilized zirconium oxide round beads of a uniform diameter. The 160 ml agitated Mini-Cer mill included a ceramic chamber and a ceramic agitator and was made by Netzsch Inc. The mill was charged with approximately 500 grams of yttrium stabilized zirconium oxide beads of a uniform diameter of variable size. The beads for these mills were provided by Norstone® Inc., Wyncote, Pa. They are highly polished and originally produced by TOSOH USA, Inc.

Particle surface area was analyzed using BET multipoint analysis on a GEMINI 2360 (Manufactured by Micromeritics® Instrument Corporation Inc., Noreross, Ga.), unless mentioned otherwise.

Micrographs of the particles were taken on an optical microscope. Micrographs are of the crystallization slurry at the end of crystallization, unless otherwise noted.

The particle size distribution of the dry cake was analyzed using laser light diffraction in a HELOS OASIS, (SYMPATEC Gbh (http://www.sympatec.com/)) machine unless otherwise noted. The same machine was also equipped with a slurry cell where a slurry of milled material or the product slurry from a crystallization could be analyzed. Standard techniques for analysis were used including the addition of lecithin to the Isopar G® carrier fluid and the application of sonication.

### EXAMPLES

### Example 1

### Compound A=Cox II Inhibitor

This series of semi-batch crystallizations demonstrate the ability to create a high surface area micro-seed by media milling and the effects of varying the amounts of micro-seed introduced during crystallization to produce final products of variable surface area and particle size. The surface area of the final product is comparable to jet milled material. Also illustrated are experiments which show that the addition of supplemental additives to the micro-seed after milling and prior to the crystallization process can increase the surface area of the resultant product, lire anti-solvent was added to cause crystallization.

Jet Milling of Compound A

Compound A was Jet milled using a typical condition ranging between 1-1.9 mm nozzles, 43-45 psig jet pressure, and 7000-21000 rpm for an 100AFG jet mill of Hosakawa Micron, Inc. The resultant surface area of the material was 2.5 m²/g.

Milling of Micro-Seed for Examples 1A-1E

On Day 0, the disc mill containing 1 mm yttrium stabilized zirconium oxide beads was flushed with 50% n-heptane and 50% toluene aid the contents of the mill were displaced for disposal by air via a positive displacement pump. To a vessel connected to the mill, 60 grams of Compound A and 1066 grams of 50:50 toluene:heptane by weight was charged. The mixture was agitated in the mill holding tank at a temperature of 25° C. The mixture was then recycled through the mill at a rate of 900 ml/min for 60 minutes. During this time, the mill was on at a tip speed of 6.8 m/s. The tank slurry was sampled at 20, 40, and 60 minutes to confirm the milling process by microscopy. Alter 60 minutes the slurry was packaged into glass jars for use later in the crystallization runs of Table 1 and 2. A jar of micro-seed slurry was filtered on a sintered glass funnel to determine the concentration of the micro-seed not dissolved in solution by drying the filler cake in a vacuum oven at 60° C. This value was reported for the basis of seed charging. The surface area of the filter cake after drying was measured by standard BET isotherm and found to be 3.4 m²/g.

Crystallizations 1A and 1B

A series of batch anti-solvent crystallizations were performed by
1) dissolving Compound A in toluene and heptane at room temperature resulting in a visually clear solution as outlined in Table 1 (“initial” charges);
2) adding a specified amount of micro-seed slurry from the milling step which initiated the crystallization due to the presence of micro-seed and additional anti-sol vent added with the micro-seed slurry;
3) adding n-heptane in portions to afford crystallization using this antisolvent. The charges were made over a 4 to 12 hour time span waiting at least 30 minutes between additions; and
4) filtering and washing the resultant slurry with sparing amounts of heptane (approximately 2-10 cake volumes) before drying at 60° C. to obtain a dry cake suitable for analysis of surface area (post-processing).

The procedure and output is described in Table 1.

TABLE 1
Anti-solvent crystallization using micro-seed from a media mill

| | Example # ID | | |
|---|---|---|---|
| | 1A | 1B | |
| | Run #1 | Run #3 | |
| time to crystallization | 1 | 2 | days since milling |
| Initial solids | 2.39 | 3.0 | g |
| Initial toluene | 27.2 | 32.4 | g |
| Initial n-heptane | 2.2 | 0 | g |
| seed concentration | 1.1 | 3.2 | wt % as solids |
| seed | 0.78 | 9.3 | g slurry |
| nominal seed level | 0.4 | 10 | wt % solids to product |
| Addition 1 | 2.7 | 1.9 | g heptane |
| Addition 2 | 4.1 | 3.2 | g heptane |
| Addition 3 | 6.8 | 5.4 | g heptane |
| Addition 4 | 9.2 | 10.0 | g heptane |
| Addition 5 | | 9.2 | g heptane |
| Surface area of dry product | 1.1 | 2 | m2/g |

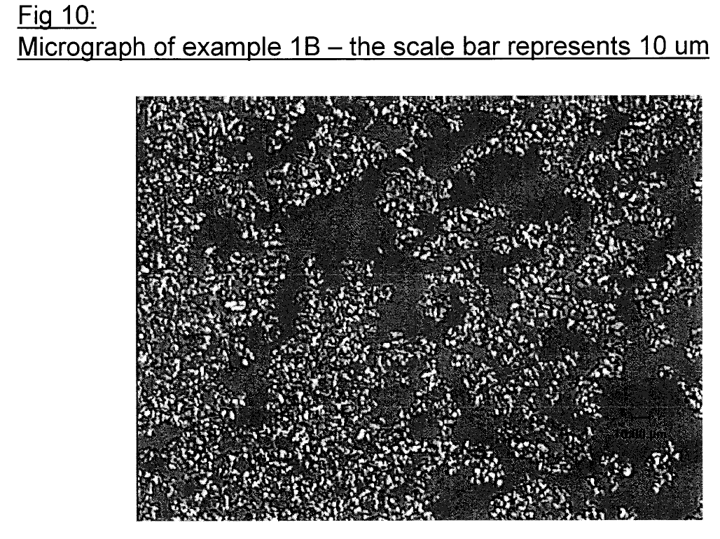

See FIG. 10 which depicts the micrograph corresponding to Example 1B. The scale bar represents 10 um.

Crystallizations 1C, 1D, and 1E

A second series of batches were conducted following the basic procedure of Examples 1A and 1B where the anti-solvent was continuously added over 12 hours (Examples 1C-1E). In Example 1D, the ionic surfactant lecithin oil (food grade) was added to the micro-seed slurry from the media mill before addition to the batch. In Example 1E, the non-ionic surfactant Triton X-100® (Sigma Aldrich) was added to the micro-seed slurry from the media mill before addition to the hatch. The addition of the non-ionic or ionic surface active agents enhanced the resultant surface area of the product obtained from those crystallizations as set forth in Table 2.

TABLE 2
Anti-solvent crystallization using micro-seed from a media mill and a
slow addition - with or without surface active agents

| | Example # ID | | | |
|---|---|---|---|---|
| | | 1D | 1E “triton | |
| | 1C | “lecithin” | X-100” | |
| time to | 3 | 4 | 4 | days since milling |
| crystallization | | | | |
| Initial product solids | 3.6 | 3.5 | 3.6 | g |
| Initial toluene | 32 | 32 | 32 | g |
| Initial n-heptane | 1.7 | 1.7 | 1.8 | g |
| seed concentration | 3.2 | 3.2 | 3.2 | wt % as solids |
| seed | 2.3 | 2.2 | 2.3 | g slurry |
| lecithin oil | | 2.2 | | g solution with seed |
| triton X-100 liquid | | | 0.185 | g solution with seed |
| nominal seed level | 2 | 2 | 2 | wt % solids to |
| | | | | product |
| time for antisolvent | 12 | 12 | 12 | hrs of addition |
| amount of | 30 | 31 | 31 | g heptane |
| antisolvent | | | | |
| Surface area of | 1.5 | 2.3 | 2.2 | m2/g |
| dry product | | | | |

### Example 2

### Compound A=Cox II Inhibitor

This series of examples demonstrate that physical slurry handling characteristics can be enhanced when supplemental additives such as anon-ionic or an ionic surfactant are added to the micro-seed wet-milling process. The supplemental additive was added to the micro-seed slurry after milling for use in the crystallization process resulting in a similar increase in product surface area as shown in Example 1D and 1E above. In addition, samples of the slurry were taken at 15 and 60 minutes to demonstrate that the milling tune can be changed as needed to afford material after crystallization of different surface area. Again, the surface area is comparable to that of jet milled material, but is produced directly by the process of the present invention.

Milling of Micro-Seed for Example 2A and 2B

On Day 0, the disc mill containing 1 mm yttrium stabilized zirconium oxide beads was flushed with 50% n-heptane and 50% toluene and the contents of the mill were displaced for disposal by air from a positive displacement pump. Sixty grams of Compound A and 1083 grams of 50:50 toluene:heptane by weight were charged to a vessel connected to the mill. A total of 10 grams of Triton X-100 was also added. The mixture was agitated in the mill holding tank at a temperature of 21° C. and the mixture was then recycled through the mill at a rate of 900 ml/min for 60 minutes. During this time the mill was on at a tip speed of 6.8 m/s. A small portion of the tank slurry was sampled at 15, 30 and 45 minutes to confirm the milling process by microscopy. After 60 minutes of milling, the slurry was packaged into glass jars for use later. A portion of a jar of micro-seed slurry was filtered on a 0.2 um filter funnel to determine the concentration of the micro-seed not dissolved in solution. The filter cake was washed with sparing amounts of the anti-solvent heptane and then dried in a vacuum oven at 60° C., The concentration of the micro-seed slurry as solids was 4.1 wt %. This concentration was approximately 30% higher than the corresponding micro-seed slurry of Example 1 where anon-ionic surfactant was not used during the milling process. This difference can be attributed to reduced physical losses in the milling system. The surface area of the filter cake after drying was measured by standard BET isotherm and found to be 3.9 m²/g.

Milling of Micro-Seed for Examples 2C and 2D

On Day 0, the disc mill containing 1 mm yttrium stabilized zirconium oxide heads was flushed with a 50% n-heptane and 50% toluene and the contents of the mill were displaced for disposal by air from a positive displacement pump. Sixty grams of Compound A and 1074 grams of 50:50 toluene:heptane by weight were charged to a vessel connected to the mill. A total of 125 grams of lecithin oil was also added. The mixture was agitated in the mill holding tank at a temperature of 20° C. lire mixture was then recycled through the mill at a rate of 900 ml/min for 60 minutes. The temperature of the outlet of the mill was 21° C. During this time, the mill was on at a tip speed of 6.8 m/s. A small portion of the tank slurry was sampled at 15, 30 and 45 minutes to confirm the milling process by microscopy. After 60 minutes of milling, the slurry was packaged into glass jars for use later. A portion of a jar of micro-seed slurry was filtered on a 0.2 um filter funnel to determine the concentration of the micro-seed not dissolved in solution. The filter cake was washed with sparing amounts of the anti-solvent heptane and then dried in a vacuum oven at 60° C. The concentration of the micro-seed slurry as solids was 4.8 wt %. This concentration was approximately 50% higher than the corresponding micro-seed slurry of Example 1 where an ionic surfactant was not used during the milling process. This difference can be attributed to reduced physical losses in the milling system. The surface area of the filter cake after drying was measured by standard BET isotherm and found to be 5.3 m²/g.

Crystallizations 2A, 2B, 2C, and 2D

A series of batch anti-solvent crystallizations were performed by
1) dissolving Compound A in toluene and heptane which resulted in a visually clear solution (“initial” charges in Table 3);
2) adding a specified amount of micro-seed slurry as shown in Table 3 after adding more non-ionic or ionic surfactant to the micro-seed;
3) adding n-heptane at a continuous rate to afford crystallization;
4) filtering and washing the resultant slurry with 2 to 10 cake volumes of heptane before drying at 60° C. to obtain a dry cake for analysis of surface area (post-processing);
   The procedure and output is described in Table 3;

| | Example # | | | | |
|---|---|---|---|---|---|
| | 2A | 2B | 2C | 2D | |
| ID | “15 min-tritonX” | “60 Min-trion X” | “15 min-lecithin” | “60-Min-lecithin” | |
| time to crystallization | 0 | 0 | 0 | 0 | days since milling |
| milling time of seed slurry | 15 | 60 | 15 | 60 | minutes |
| Initial product solids | 3.6 | 3.6 | 3.5 | 3.5 | g |
| Initial toluene | 32 | 32 | 32 | 32 | g |
| Initial n-heptane | 1.7 | 1.7 | 17 | 1.8 | g |
| seed concentration | 4.1 | 4.1 | 4.8 | 4.8 | wt % as solids |
| seed | 1.8 | 1.6 | 2.2 | 2.2 | g slurry |
| extra lecithin oil | | | 2.2 | 2.2 | g solution with seed |
| extra triton X-100 liquid | 0.14 | 0.14 | | | g solution with seed |
| nominal seed level | 2.0 | 2.0 | 3.0 | 3.0 | wt % solids to product |
| cystallization temperature | 25 | 25 | 27 | 27 | hrs of addition |
| time for antisolvent | 12 | 12 | 12 | 12 | hrs of addition |
| amount of antisolvent | 30 | 30.3 | 30 | 30 | g heptane |
| Surface area of dry product | 2.0 | 2.2 | 1.7 | 2.2 | m2/g |

### Example 3

### Compound B=Cox II Inhibitor

This series of examples demonstrate the ability to replace pin milling for a compound known to exhibit “meltback”. The form of the crystal is controlled throughout the process even though four other possible crystalline forms of Compound B are known. The crystallizations were performed at elevated temperature. This example demonstrates that the surface area can be controlled by the addition of different levels of micro-seed.

Pin Milling of Compound B

Compound B was Pin milled for pharmaceutical use using typical conditions for an Alpine® UPZ160 mill (Hosakawa) and with a high process nitrogen flow. This compound is difficult to mill due to the low melting point of the compound. Cold nitrogen at 0° C. and 40 SCFM (standard cubic feet per minute) was applied as a pin rinse of the mill during processing to keep the processing temperature below the melting point of the compound. Milling was not possible without this extra step. The resultant surface area of the material was 0.9 m²/g.

Milling of Micro-Seed for Example 3A and 3B

On Day 0, the disc mill containing 1 mm yttrium stabilized zirconium oxide beads was flushed with 50% n-heptane and 50% toluene and the contents of the mill were displaced for disposal by air from a positive displacement pump. Sixty grams of Compound B and 1066 grams of 50:50 toluene:heptane by weight were charged to a vessel connected to the mill. The mixture was agitated in the mill holding tank at a temperature of 25° C. and the mixture was then recycled through the mill at a rate of 900 ml/min for 60 minutes. During this time the mill was on at a tip speed of 6.8 m/s. The temperature of the mill outlet was 25° C. A small portion of the tank slurry was sampled at. 15, 30 and 45 minutes to confirm the milling process by microscopy. After 60 minutes of milling in total the slurry was packaged into glass jars for use later. From one jar of micro-seed slurry, 122.8 g was filtered on a filter funnel and the filter cake was washed with sparing amounts of the anti-solvent heptane. A total of 9.7 grams of wet cake was collected. This was then dried in a vacuum oven at 60° C. The surface area of the filter cake after drying was measured by standard BET isotherm and found to be 5.7 m²/g.

Crystallizations 3A and 3B

A series of batch anti-solvent crystallizations were performed by
1) dissolving Compound B in toluene and heptane at 50° C. m an 50 ml agitated vessel which resulted in a visually clear solution, denoted as the “initial” charges in Table 4;
2) adding a specified amount of micro-seed slurry from the milling step which initiated the crystallization due to the presence of micro-seed and additional anti-solvent added with the micro-seed slurry:
3) adding n-heptane at a continuous rate to afford crystallization;
4) filtering the resultant slurry at room temperature, and washing with 2 to 10 cake volumes of heptane before drying at 60° C. to obtain a dry cake for analysis of surface area.
   The procedure and output is described in Table 4:

| | Example # ID | | |
|---|---|---|---|
| | 3A | 3B | |
| | “0.36 wt %” | “10 wt %” | |
| time to crystallization | 1 | 1 | days since milling |
| milling time of seed slurry | 60 | 60 | minutes |
| Initial product solids | 4.8 | 4.8 | g |
| Initial toluene | 32 | 40 | g |
| Initial n-heptane | 2.4 | 0.0 | g |
| seed | 0.5 | 14.7 | g slurry |
| nominal seed level | 0.4 | 10 | wt % solids |
| | | | to product |
| crystallization temperature | 50 | 50 | C. |
| time for antisolvent | 12 | 12 | hrs of addition |
| amount of antisolvent | 30 | 40 | g heptane |
| Surface area of dry product | 0.6 | 1.1 | m2/g |

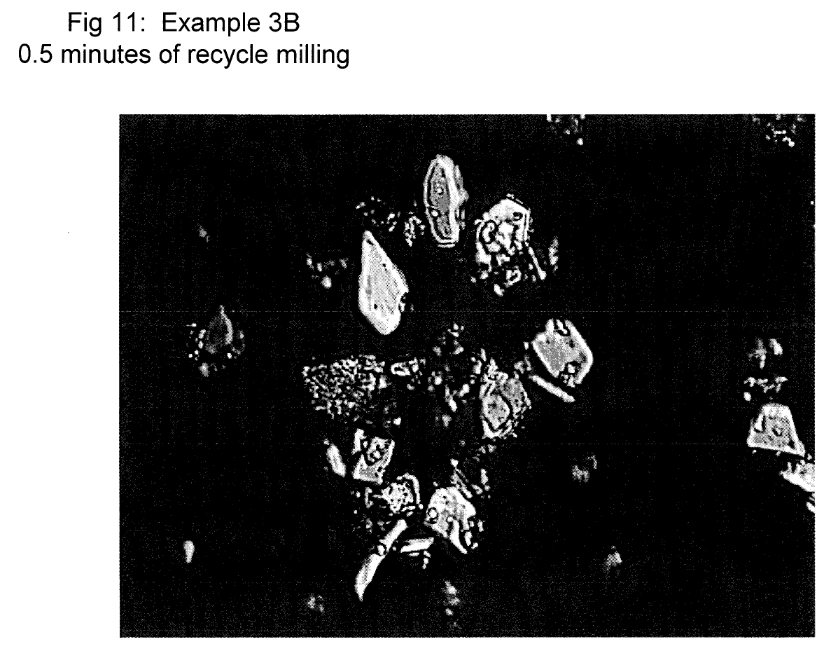

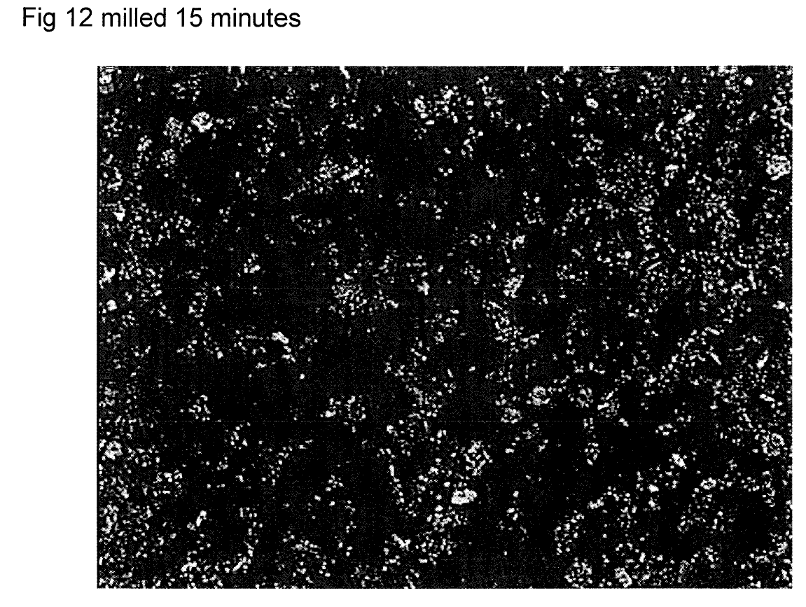

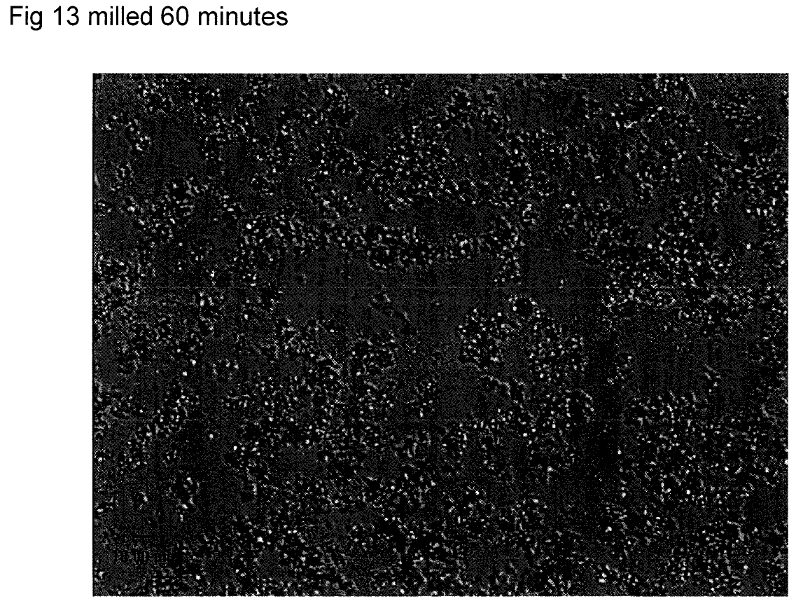

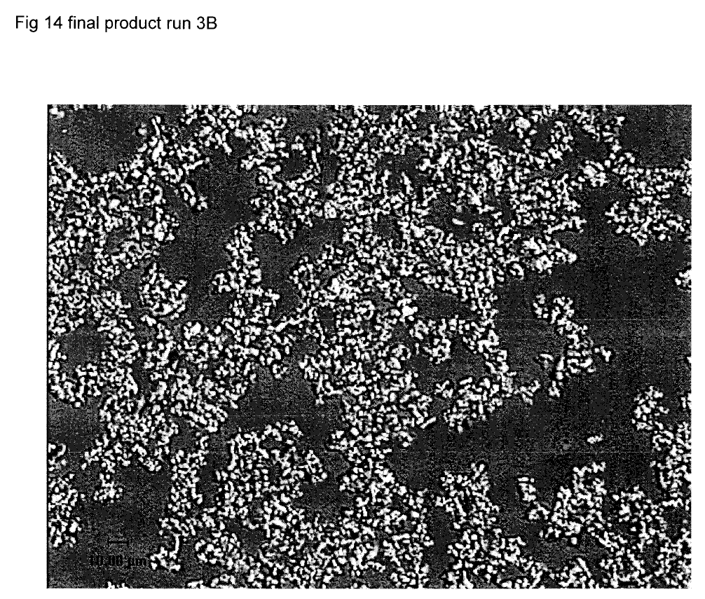

FIG. 11 is a micrograph of the micro-milling slurry of Example 3B after 0.5 minutes of recycle milling. FIG. 12 is a micrograph of the micro-milling slurry of Example 3B after 15 minutes of recycle milling. FIG. 13 is a micrograph of the micro-milling slurry of Example 3B after 60 minutes of recycle milling. FIG. 14 depicts the micrograph corresponding to the final product after crystallization of Example 3B. The scale bar represents 10 μm.

### Example 4

### Compound C=BK1 Antagonist

This series of examples demonstrates that multiple pharmaceutical classes can be accommodated using the methods of the present invention. It also demonstrates that the surface area of the final product can be controlled by using different size micro-seed. The micro-seed size can be altered using different amounts of milling time. The seed particles generated by the milling step in this example are above 1 um in size. Compound C has a low melting point and the MMC process is useful to avoid “meltback” during dry milling. Cold nitrogen must be applied as a pin rinse of the pin mill to enable milling a significant quantity of material.

Milling of Micro-Seed for Example 4A and 4B

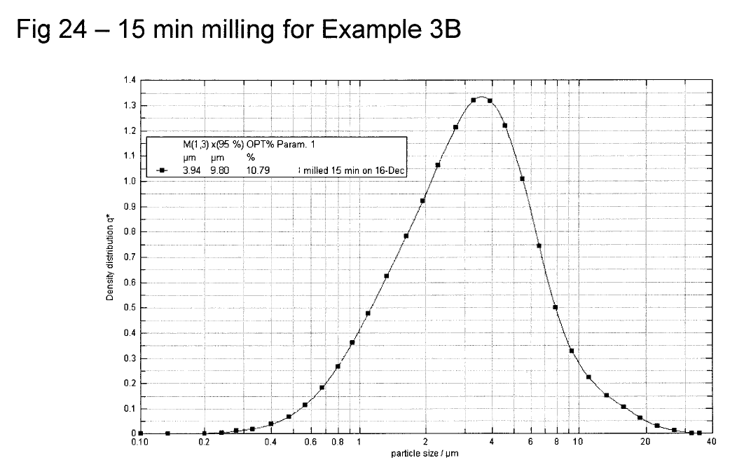

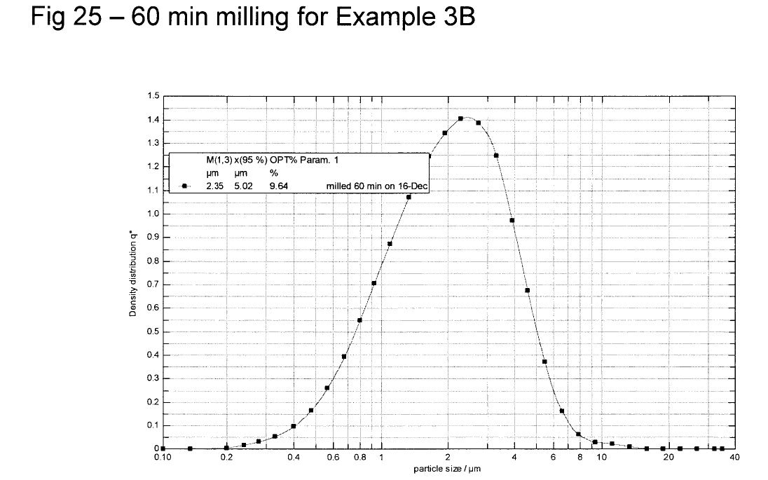

On Day 0, the disc mill containing 1 mm yttrium stabilized zirconium oxide beads was flushed with 50% n-heptane and 50% toluene by weight and the contents of the mill were displaced for disposal by air from a positive displacement pump. Sixty grams of Compound C and 1066 grams of 50:50 toluene:heptane by weight were charged to a vessel connected to the mill. The mixture was agitated in the mill holding tank at a temperature of 19° C. and the mixture was then recycled through the mill at a rate of 900 ml/min for 60 minutes. During this time the mill was on at a tip speed of 6.8 m/s. The temperature of the mill outlet was 20° C. A small portion of the tank slurry was sampled at 0, 15, 30 and 45 minutes to confirm the milling process by microscopy. After 60 minutes of milling in total, the slurry was packaged into glass jars for use later. Tire slurry samples were analyzed on the SYMPATEC® light diffraction wet cell analyzer using lecithin and 120 seconds of sonication in ISOPAR G® FIGS. 24 and 25 demonstrate the particle size distribution of the micro-seed. For the micro-seed milled 15 minutes, the mean particle size by volume is 3.9 um and 95% of the particles by volume are less than 9.8 um. For the micro-seed milled 60 minutes, the mean particle size by volume is 2.35 um and 95% of the particles by volume are less than 5.2 um indicating a sharper particle size distribution using micro-seed milled longer. A portion of the micro-seed slurry from 15 minutes and 60 minutes of milling was filtered and washed with heptane and dried at 60° C. as in the previous examples. After drying the surface area of the filter cakes was measured by standard BET isotherm and found to be 4.6 m2/g for 1.5 minutes of milling and 6.6 m2/g for 60 minutes of milling. This data demonstrates that micro-seed size and surface area can be controlled by process parameters.

Crystallizations 4A and 4B

Two batch anti-solvent crystallizations were performed by
1) dissolving Compound C in toluene and heptane at 43° C. in a 75 ml vessel agitated by overhead stirrer which resulted in a visually clear solution (the “initial” charges);
2) the slurry was cooled to 40° C. to generate a supersaturated solution without solids forming as verified visually by in-situ Sight backscattering;
3) adding a specified amount of micro-seed slum from the milling step;
4) adding n-heptane at a continuous rate to afford crystallization; and
5) filtering the resultant slurry at room temperature, and washing with 2 to 10 cake volumes of heptane before drying at 60° C. to obtain a dry cake for analysis of surface area.
   The procedure and output is described in Table 5:

| | Example # ID | | |
|---|---|---|---|
| | 4A | 4B | |
| | “15 min” | “60 min” | |
| time to crystallization | 0 | 0 | days since milling |
| milling time of seed slurry | 15 | 60 | minutes |
| Initial product solids | 1.4 | 1.4 | g |
| Initial toluene | 40 | 40 | g |
| Initial n-heptane | 0.0 | 0.0 | g |
| seed | 1.1 | 1.1 | g slurry |
| nominal seed level | 2.5 | 2.5 | wt % solids to |
| | | | product |
| cystallization temperature | 40 | 40 | C. |
| time for antisolvent | 12 | 12 | hrs of addition |
| amount of antisolvent | 40 | 40 | g heptane |
| Surface area of dry product | 0.7 | 1.0 | m2/g |

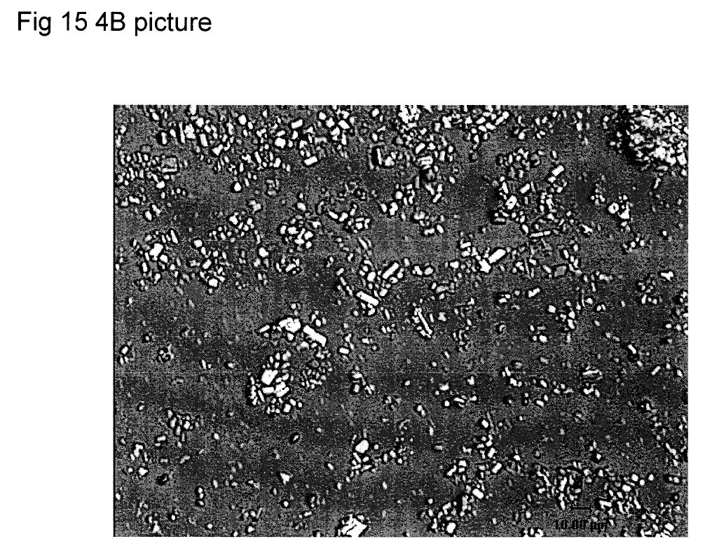

FIG. 15 depicts the micrograph of the final product of Example 4B.

### Example 5

### Compound D=Bisphosphonate

This example demonstrates that particle sizes obtained by conventional crystallization followed by pin milling of a dry cake can be replicated by the MMC process. This example also demonstrates a temperature cooldown crystallization and another drug class. Different sized media beads were used and the process was aqueous based.

Conventional Approach

Compound D was dissolved in water at 100 g/l at 60° C. The compound was cooled to 0° C. and distilled to 200 g/l simultaneously to provide a crystallized product. The material was filtered, dried and pin milled using typical pin milling conditions. The pin milling of this product is especially difficult. A functional mill was only maintained when the mill was shut down and the pins cleaned after each 40 kg of material processed. This process yielded a 5-40 um product as analyzed visually by micrograph.

Milling of Micro-Seed for Example 5

On Day 0, the disc mill was charged with 1890 g of 1.5 mm yttrium stabilized zirconium oxide beads and flushed with deionized water. The contents of the mill were displaced for disposal by air from a positive displacement pump. Thirty-four grams of Compound D and 207 grams of deionized water by water weight were charged to a vessel connected to the mill. The mixture was agitated in the mill holding tank while being recycled through the mill at a rate of 630 ml/min for 10 minutes. During this time the mill was on at a tip speed of 6.8 m/s. The mill outlet temperature was 20° C. A small portion of the tank slurry was sampled at 0 and 5 minutes to confirm the milling process by microscopy. After 10 minutes of milling, the slurry was packaged into glass jars for use later. A micrograph of the micro-seed indicated a size larger with 1.5 mm beads than runs with 1.0 mm heads.

Crystallizations 5

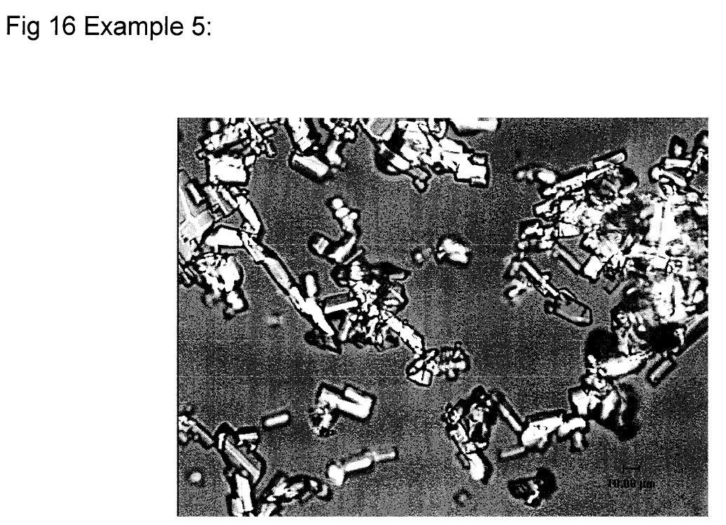

On Day 0, a temperature cooldown crystallization was performed by dissolving 14.0 g Compound D in 95 g water in an 75 ml vessel agitated by overhead stirrer which resulted in a visually clear solution. The temperature of the jacket enclosing the vessel was held at 66° C. for this dissolution. The slurry was cooled by placing 64° C. on the jacket to generate a supersaturated solution without solids forming. Supersaturation was verified visually and by in-situ light backscattering. A total of 4.0 grams of slurry micro-seed from the milling step was added and the jacket temperature was changed to 64° C. The jacket was then cooled from 61 to 48° C. over 4 hours and from 48 to 20° C. over 7 hours. A micrograph of the micro-seed slurry was analyzed for visual particle size analysis. The mean length was 17 um and the mean width was 8 um. This size mimics that needed for the pharmaceutical application. FIG. 16 is a micrograph of the final product of Example 5.

### Example 6

### Compound F=Serotonin Antagonist

This series of examples demonstrate that the MMC process can meet the bioavailability of the product produced by a AFG jet mill as measured by canine blood plasma levels. This series of examples further demonstrates the utility of a supplemental energy device placed in the crystallization vessel (in this case a sonicator) to promote a product with smaller particle size (higher surface area). Example 6 demonstrates that smaller beads in the milling process lead to higher surface area micro-seed aid higher surface area of the product when the same charge of micro-seed was employed. This example demonstrates that the use of higher level of seed, here 20%, can enhance the surface area of the product. The example is a semi-continuous process with mixed aqueous organic solvents. Compound F is known to have several polymorphs and the process in accordance with the present invention produced the desired polymorph. This demonstrates the feasibility of the MMC process for pharmaceutical processing.

AFG Milling

Material was 100AFG milled with 1 mm nozzles, 50 psig jet pressure. 9000-18000 rpm and the surface area was 0.6 m²/g.

Milling of Micro-Seed #1 for Example 6

On Day 0, the disc mill containing 1890 grams of 1.5 mm yttrium stabilized zirconium oxide beads was flushed with 60% isopropanol (IPA) and 40% deionized water by volume. The contents of the mill were displaced for disposal by air from a positive displacement pump. To a vessel connected to the mill, were charged 18.5 grams of Compound F and 220 grams of 60/40 IPA/Water. The mixture was agitated in the mill holding tank while being recycled through the mill at a rate of 600 to 900 ml/mill for 15 minutes. During this time the mill was on at a tip speed of 6.8 m/s and the mill outlet temperature was below 30° C. A small portion of the tank slurry was sampled at 0.5, and 10 minutes to confirm the milling process by microscopy. After 15 minutes of milling, the slurry was packaged into glass jars for use later.

Milling of Micro-Seed #2 for Example 6

The procedure of Milling #1 above was duplicated except 1894 grams of 3.0 mill yttrium stabilized zirconium oxide beads were used as media.

Semi-Continuous Crystallization

Semi-continuous crystallization was accomplished by the simultaneous addition of the micro-seed slurry concentrate and the antisolvent for the specified charge time. The solvent ratio was maintained during the addition of the concentrate. The charges were made through a 22 gauge needle below the liquid-gas surface near the agitator on opposite sides of the vessel. The 75 ml vessel employed an overhead stirrer for agitation and an 8 mm sonication probe placed below the liquid-gas surface. Where noted in Table 7, the sonication probe was on during the crystallization at a power of approximately 10 watts. For the runs using Media milled seed #2, additional water was added at the end of the batch concentrate addition at the same rate when charged with concentrate to change the solvent ratio from 4:3 to 1:2 IPA:water. This was done to improve yield approximately 5% by lowering the mother liquor losses and did not impact the particle size significantly. Post processing comprised filtration of the slurries at room temperature via vacuum and drying with air or drying in a vacuum oven at 40° C.

The yield of Example 6C of Table 7 was quantified to be 85%. This run was shown by X-Ray diffraction to yield the desired hemi-hydrate form.

Run Summary Table 7:

| | | | | ratio | | | |
|---|---|---|---|---|---|---|---|
| | | charge | constant | IPA:H20 | | | |
| | seed % | hr | sonication | (γ · ml loss) | SA | mv (um) | 95% < (um) |
| Media milling #1 (1.5 mm beads) | | | | 4:3 | 2.3 | 4.1 | 10.2 |
| 6A | 10 | 6 | none | 4:3 | 1.9 | 12.1 | 39.8 |
| 6B | 10 | 3 | yes | 4:3 | 2.2 | 7.7 | 17.4 |
| Media milling run #2 (1.0 mm beads) | | | | 4:3 | 3.5 | 3 | 7.6 |
| 6C | 10 | 3 | yes | 1:2 | 2.3 | 6.5 | 18 |
| 6D | 20 | 3 | yes | 1:2 | 2.6 | 6 | 10.3 |

Post Formulation and Use

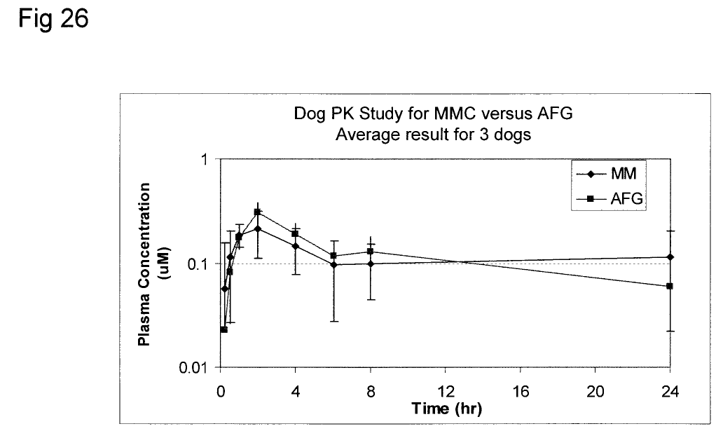

The solid product of Example 6C and the AFG milling sample were formulated in a side by side study into direct filled capsules using conventional pharmaceutical ingredients. The area under the curve (AUC in 24 hours) for Dogs of MMC Example 6C was compared versus AFG milled material indicating equivalent bio performance was obtained. The results are provided in FIG. 26.

### Example 7

### Compound G=DP IV Inhibitor

This example demonstrates that large particles (>50 um) can be made consistently by the MMC process of the present invention. The particle size can be tailored using different seed loads.

Media Milling

On Day 0, the KDL media mill was flushed with 80/20 IPA/water and pumped dry. A slurry of Compound G at 100 mg/g in 80/20 IPA/water by weight was fed through the mill in recycle mode at a rate of 300 mls/min for 120 minutes. The resulting particle size of the micro-seed had a mean size of 4.7 um as measured by light diffraction.

Crystallization

A series of crystallizations were made using the media milled micro-seed of Example 7. In these crystallizations, the seed amount was varied. A batch of Compound G at 220 mg/g in 70/30 by weight IPA/Water was heated to over 70° C. to dissolve the solids. A visually clear solution was obtained. The batch was cooled to 65 to 67° C. to create supersaturation. The batch was seeded with the level of micro-seed as indicated in Table 8 (grams of dry product added to the seed slurry versus that in the batch). The batch was aged 3 hours and cooled to room temperature over 5 hours, Isopropyl alcohol anti-solvent was charged over a period of 15 to 30 minutes to reach 80/20 IPA/water by weight. The batch was aged 1 hour and vacuum filtered and vacuum dried in an oven at 45° C. The particle size was analyzed via a Microtrac particles size light diffraction using 30 second sonication at approximately 30 watts in the wet state. The following results were obtained.

TABLE 8

| Run Number | Day | Seed load (%) | Mv (um) | 95% < (um) |
|---|---|---|---|---|
| 7A | 5 | 0.5 | 77 | 179.1 |
| 7B | 13 | 0.5 | 72 | 158.5 |
| 7C | 10 | 2 | 52 | 120.5 |

### Example 8

### Compound D=Bisphosphonate

The example demonstrates scale up of the MMC process and the utility of a recycle loop to enhance the mixing characteristics of a vessel upon scale up. This example further demonstrates that a higher intensity energy device placed in the recycle loop (here a static mixer) can enhance the surface area achieved for the final product. This series of examples demonstrates a profile comparable to pin milled product.

Pin Milling

Compound D was crystallized. The product was pin-milled and the resulting particle size was measure by light diffraction as 18.7 um with 95% less that 50 um. The surface area, was 0.53 m²/g.

Milling of Micro-Seed for Example 8

A series of media milling runs were made to supply micro-seed for the crystallization. On Day 0, the disc mill was charged with 1.5 mm yttrium stabilized zirconium oxide beads and then flushed with deionized water. The contents of the mill were displaced for disposal by air from a positive displacement pump. Slurries at the equivalent of 100 grams per 1 liter deionized water concentration were charged to a vessel connected to the mill. The mixture was agitated in the mill holding tank while being recycled through the mill at a rate of 900 ml/min. During this time the mill was on at a tip speed of 6.8 m/s and the mill outlet temperature was 25° C. After milling, the slurry was packaged into glass jars for later use.

Crystallizations 8

A series of temperature cooldown crystallizations were performed by dissolving 250 grams of Compound D in 2500 g deionized water in an agitated vessel using an overhead stirrer. The temperature of the jacket enclosing the vessel was increased and the batch temperature was raised to 60-62° C. to dissolve the batch to a visually clear solution. The slurry was cooled to 52° C. to generate a supersaturated solution without solids forming as verified visually. A total of 115 milliliters of micro-seed slurry was added to the vessel via the top of the reactor and aged at 52 to 53° C. for 30 minutes. The batch was cooled to 5° C., aged for at least 1 hour and then filtered cold using a vacuum filter and vacuum dried at 45° C.

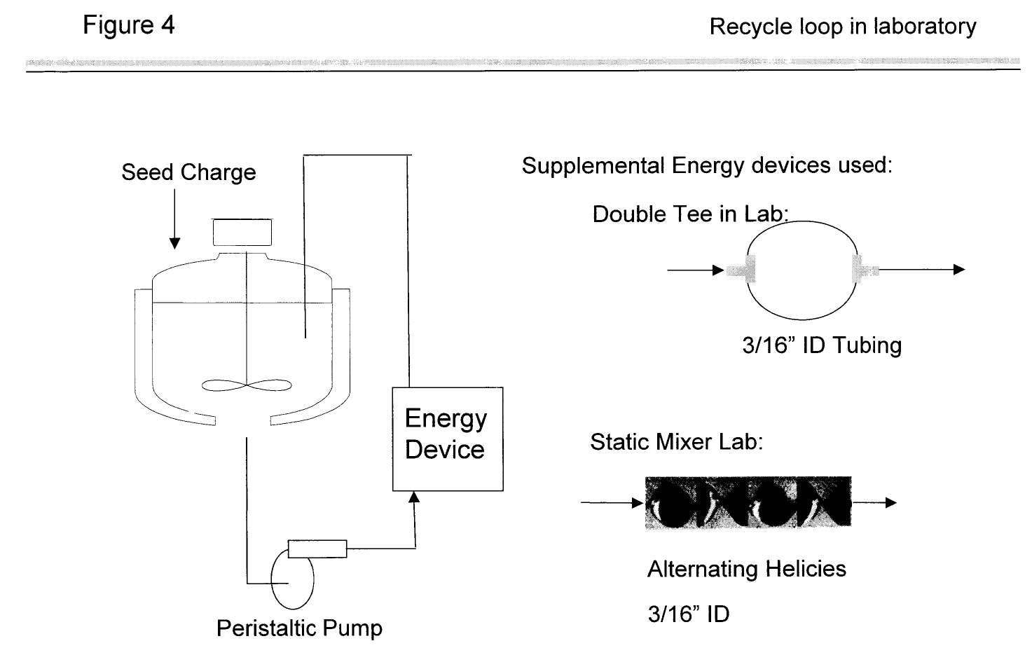

Based on the concentration of product in the mother liquor at the final solvent composition, a yield of at least 80% is expected for this set of examples. The particle surface area was analyzed by BET isotherm and light diffraction. The particles of run 8A were highly agglomerated and exceeded the capability of the light diffraction machine to measure. Addition of a recycle loop as depicted in FIG. 4 enhanced the surface area of the product. Addition of a static mixer which is a higher energy device in the recycle loop lead to higher surface area comparable to that produced by pin milling the dry product.

TABLE 9

| Example # | | |
|---|---|---|
| 8A | 8B | 8C |

| | | | |
|---|---|---|---|
| Milling | | | |
| grams product | 220 | 220 | 50 |
| grams of water | 2200 | 2200 | 500 |
| time for milling process, min | 30 | 45 | 15 |
| mill outlet temp | 25 | 25 | 25 |
| seed sonicated before use | — | — | no |
| Day Used after milling | 5 | 1 | 2 |
| Crystallizer Set Up | | | |
| ag rate | 300 | 350 | 450 |
| ag diameter, cm | 5 | 6 | 6 |
| recycle rate, ml/min | — | 900 | 450 |
| Energy Device | — | double tee | static mixer |
| Conditions | | | |
| batch size, liters | 1 | 2.5 | 2.5 |
| cooldown time, hours | 6 | 10 | 3 |
| seed load, wt % | 2 | 3 | 3 |
| Surface area of product, m2/g | 0.12 | 0.36 | 0.46 |
| Mean particle Size (microns) | >75 um | 23 | 15 |
| 95% < um | | 50 | 29 |
| % < 10 um | | 18 | 30 |

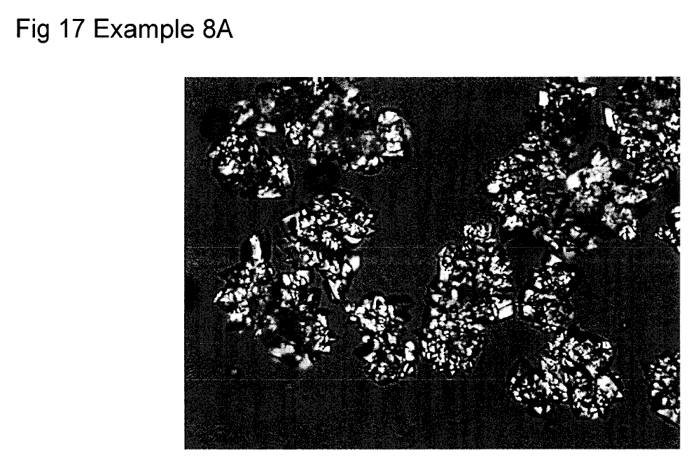

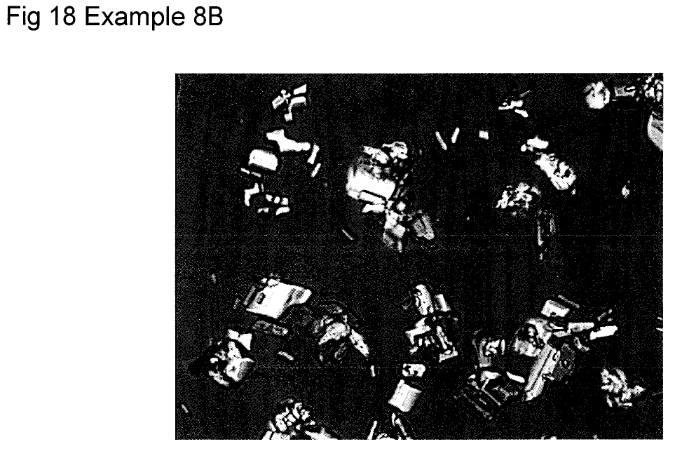

The results of Example 8A demonstrated that the equipment chosen to scale up the MMC process can alter the product results. Adding a recycle loop to a vessel to aid in mixing is an embodiment of the present invention. Furthermore, Example 8C demonstrates that adding a supplemental energy device can provide a higher energy in the recycle loop thereby yielding a product of enhanced surface area. The surface area of Example 8C matches that produced by pin milling. The crystallizations produced without a recycle loop or supplemental energy device lead to visually agglomerated material of relatively lower surface area and larger particle size as shown in FIGS. 17 and 18.

### Example 9

### Compound E=Lipid-Lowering Compound

This example demonstrates semi-continuous crystallization with antisolvents where multiple charge times for antisolvent and concentrate can be accommodated. Sonication is shown useful to enhance surface area of the product. Here, smaller beads of 0.8 mm were used to demonstrate that a range of beads sizes can be utilized in accordance with the process of the present invention.

Conventional Drymilling Approach

Compound E was jet milled. The resultant surface area specification was 1.4 to 2.9 m²/g for the product.

Milling of Micro-Seed for Example 9

On Day 0, the disc mill was charged with 0.8 mm yttrium stabilized zirconium oxide beads in the dry state. To a vessel connected to the mill was charged 1000 ml of 60/40 MeOH/water and then 60 grams of Compound E and then 0.2 grams of butylared hydroxy anisole (BHA) as a supplemental additive for performance of the product The mixture was agitated in the mill holding tank while being recycled through the mill at a rate of 900 ml/min for 30 minutes. During this time the mill was on at a tip speed of 6.8 m/s and the mill outlet temperature was 21° C. A small portion of the tank slurry was sampled at 0 and 30 minutes to confirm the nulling process by microscopy. After 30 minutes of milling in total, the slurry was packaged into glass jars for later use. The mean micro-seed size was determined to be about 2 um.

Crystallizations 9A, 9B, 9C, 9D

Semicrontinuous anti-solvent crystallizations were performed by:
1) creating concentrate by dissolving 60 g of Compound E in 1 liter of methanol. A total of 0.2 grains of butylated hydroxy anisole was added to this stream in order to prevent oxidation of the product;
2) creating micro-seed bed by charging 5 ml of micro-seed slurry from milling and adding 5 ml of 60/40 Methanol water by volume. The charges were made to a 1.00 ml agitated vessel at 600 RPM with a 22 mm diameter blade;
3) simultaneously charging the 56 milliliters of concentrate and 36 milliliters of deionized water anti-solvent were charged to the vessel via separate syringe pumps;
4) aging the batch for 1 hour at room temperature. Sonication at approximately 10 watts of power was applied directly into the crystallizer during the concentrate additions and 1 hour age period using a 8 mm probe (DG30 manufactured by Telesonics);
5) filtering the resultant slurry at room temperature before vacuum drying at 45° C. to obtain a dry cake for analysis of surface area. The particle size was measured by dry solids light diffraction.

Based on the concentration of product in the mother liquors at the final solvent composition, a yield of at least 80% is expected for this set of examples. The runs were made using identical reactor systems.

The procedure and output is described in Table 10:

| | Example # | | | | |
|---|---|---|---|---|---|
| | 9A | 9B | 9C | 9D | |
| Day | 1 | 1 | 2 | 2 | days since milling |
| Addition time of | 3 | 3 | 10 | 10 | hours |
| concentrate | | | | | |
| Sonication during | yes | no | yes | no | |
| addition | | | | | |
| nominal seed level | 10 | 10 | 10 | 10 | wt % solids to product |
| cystallization | 20 | 20 | 20 | 20 | C. |
| temperature | | | | | |
| Surface area of dry | 2.6 | 2.1 | | | m2/g |
| product | | | | | |
| Mean particle Size | 6.4 | 11.8 | 7 | 10.1 | microns (um) |

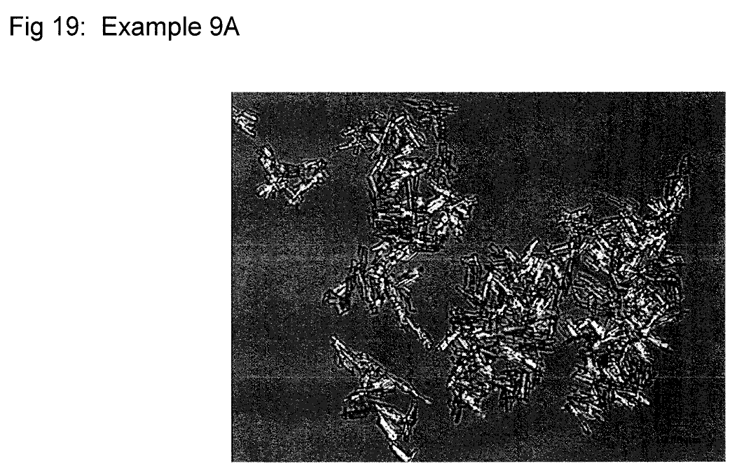

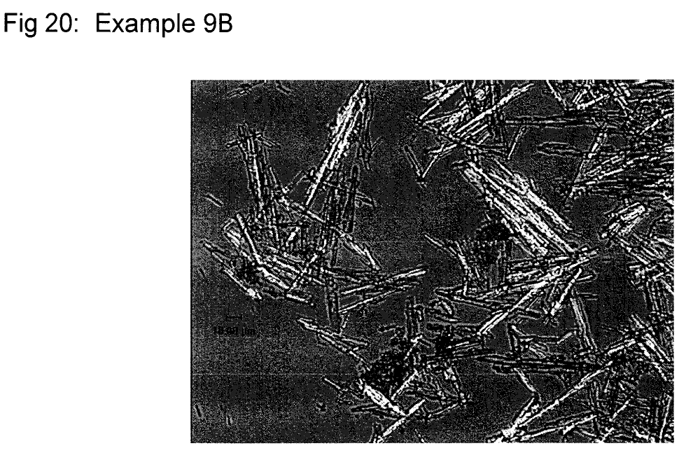

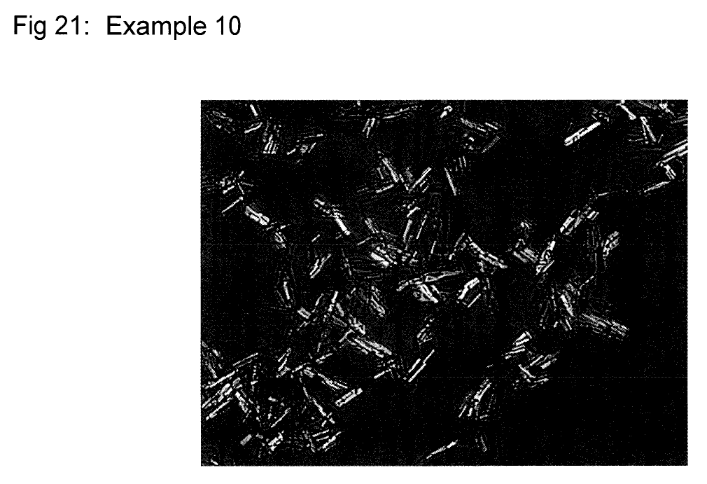

Micrographs of the product of Example 9A and 9B are shown in FIGS. 19 and 20, respectively. The products are similar except for the length of the individual crystals. FIG. 19 can be compared to FIG. 21 where the process was scaled up using less sonication power and a longer addition time to limit any nucleation.

### Example 10

### Compound E=Lipid-Lowering Compound

This example demonstrated that the process of the present invention was amenable for scale up to a commercial production volume level for specially chemicals. Here a scale of 15 kg of product is produced in one batch using a semi-continuous batch method. A larger scale emulation of the recycle loop is described which produced a successful scale up. The recycle rate corresponded to 18 minute batch turnover time, practical rate for a large scale manufacturing process. The sonication power density was approximately 0.7 W/kg of batch, a practical level for a large scale manufacturing process. The crystallization product was post processed using conventional manufacturing equipment. As with many pharmaceuticals, the product was oxygen sensitive and all streams were degassed using either nitrogen flow or vacuum application. The supplemental additive, butylaled hydroxy anisole (BHA), was used as a product stabilizer.

Milling of Micro-Seed for Example 10

A total of 1.49 kg of Compound E unmilled pure, 9.3 kg of deionized water, 14 kg of methanol and 8.14 g of BHA were charged to a jacketed 30 liter glass vessel equipped with an agitator to blend the vessel contents. The slurry was charged with nitrogen to degas the solution and a nitrogen sweep was used throughout the milling process to keep the system inert. A large quantity of solids was charged and the material demonstrated clumping during wetting. In order to declump the material, a ⅜″ ID recycle line was connected to the vessel which contained a rotor stator mill (IKA® Works T-50 with coarse teeth). The batch was recycled through the wet mill for 30 minutes to break up the large chunks of solid. The IKA Works mill was used as the pump to recycle the batch volume at least twice during this step. The recycling step did not reduce the particle size of the product significantly.

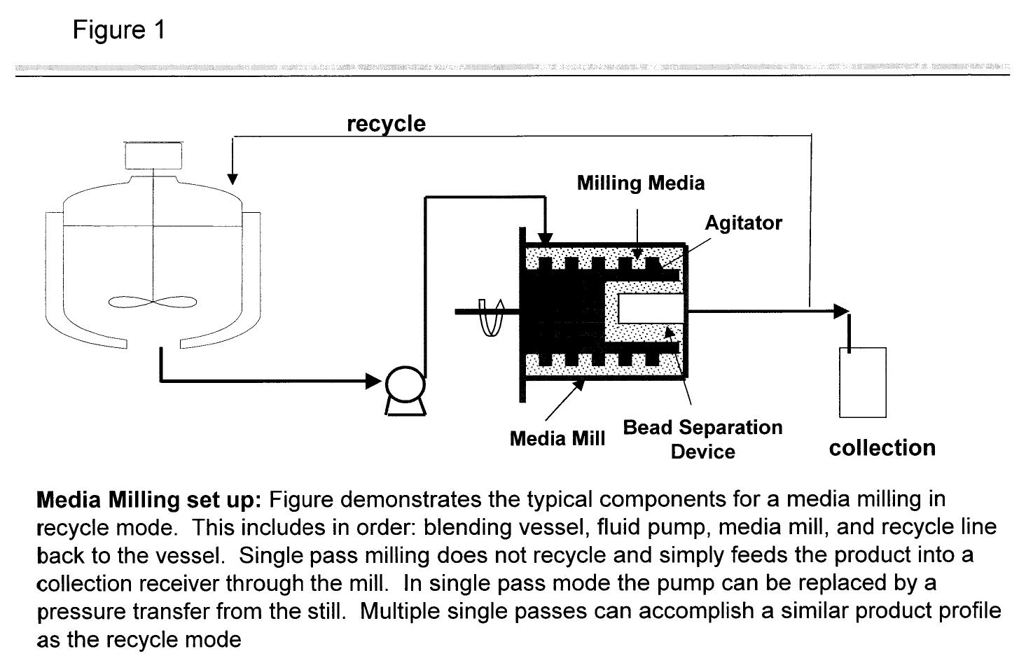

To mill the batch to micro-seed, a second recycle line was constructed as in FIG. 1. The pump was a peristaltic Masterflex aid the mill was a Netzsch media mill model number “Minicer”. The mill was charged with 135 ml of 1 mm yttrium stabilized zirconium oxide beads (approximately 500 grams). The batch slurry was then recycled through the Minicer mill at a rate of 300 ml/rain rate using the Masterflex® volumetric pump. The mill was run at 2202 rpm, corresponding to a 6.8 m/s tip speed. The mill and the batch vessel were cooled by glycol baths to maintain the batch slurry temperature below 25° C. throughout the milling process. The batch slurry was milled for a total of 41 hours. The milled slurry was aged overnight at room temperature, then discharged though the media mill into a poly drum for use within the next 3 hours. The milled slurry was the micro-seed stream. A portion of the slurry was filtered on a 0.2 um filter and analyzed after diving in a vacuum oven at 40° C. At the time of discharge of the slurry, the surface area of the milled solids was 4.05 m²/g with a volume mean particle size of 2.1 μm and 95% of the particles less than 4.8 μm by volume. A Helos analyzer was used.

Crystallization for Example 10

Recycle hop setup: The larger scale equipment is similar to the set up of FIG. 3 except that an inline laser backscattering probe was used to measure the chord length of the particles in the slurry in real time and the seed was charged before the first mixing device. The recycle loop from the bottom of the 100 gallon stirred tank consisted of:

1) a diaphram pump;
2) a focused beam reflectance measurement probe for chord length monitoring:
3) ⅜″ valve port for sampling and charging seed slurry as needed;
4) a rapid mixing device connected to a pump for deionized water antisolvent addition from a claim:
5) an energy device consisting of a radial sonicator horn of 2″ diameter and 22″ long in a 2 liter flow through cell. The sonicator was manufactured by Telesonics and was powered by a generator of 2000 W:
6) a rapid mixing device connected to a pump for batch concentrate addition from a drum;
7) a mass meter to measure the recycle rate of slurry;
8) pipe returning to the main crystallizer which was 13/16″ internal diameter:

Antisolvent stream: To a vessel previously cleaned and flushed with deionized water, a total of 250 kg of deionized water was charged. The deionized water was degassed using several vacuum and nitrogen pressure purges. The water was drummed in 50 gallon drums and kept closed till use. This stream was the antisolvent stream.

Batch stream: To a vessel flushed with methanol, a total of 14 kg of Compound E active pharmaceutical ingredient (API), 144 kg of methanol (previously degassed), and 80 g BHA inhibitor was charged. Compound E concentrate was drummed into 50 gallon drums and kept closed until use. This was the hatch stream.

Micro seed slurry make up: A total of 36 kg of previously made up 60/40 vol./vol. methanol/water solution was charged to a 100 gallon crystallizer. The solution was recycled at approximately 25 kg/min using the recycle loop. The sonicator radial probe was set at 350 W power, and the Lasentec® FBRM probe was turned on for information. The micro-seed slurry described in this example above was charged to the recycle loop via the ⅜″ seed charge port Tee and the seed bed was recycled for 15 minutes with sonication at 20 to 25° C. This was the micro-seed for the batch.

Crystallization charges: The vessel agitator was 22″ in diameter and was spinning at 3 m/s for the crystallization. A total 129 kg of deionized water was charged to the micro-seed, along with 168 kg of Compound E in methanol batch concentrate, over 10 hrs time simultaneously at a constant charge rate. Throughout the crystallization the batch was kept at 20 to 25° C. while continuous sonication at 350 W was applied. Samples were taken after 1, 3, 6 and 10 hr addition to confirm the crystallization progress. After simultaneous addition was completed, 84 kg of deionized water was charged at a constant charge rate over two hours with sonication at 20 to 25° C. The addition of extra water antisolvent was made to increase the yield by lowering the solubility for the product. The charges were made slowly to promote growth of the crystals versus nucleation.

After the deionized water charge, the batch was aged with sonication at 20 to 25° C. for 1 hour to ensure complete growth of the crystals. A picture of the crystal slurry was collected using an optical microscope as indicated in FIG. 21. FIG. 21 demonstrates that the particles were monodispersed with no small particles due to uncontrolled nucleation present. The recycle loop was turned off and the batch was aged at 20 to 25° C. overnight Post processing by filtration and drying of the batch followed.

Post Processing for Example 10

Filtration and drying: After an overnight age in the vessel, the batch was filtered at room temperature. A total of 385 kg of mother liquors with a Compound E concentration of less than 1 mg/g were collected. A total of 20 kg of previously made up 50/50 v/v methanol/water was charged to the crystallizer via a spray ball in order to wash the walls of the vessel into the batch filter and wash the product in the filter. A total of 40 kg of wash and residual mother liquors was collected. After filtration and application of nitrogen pressure to the cake for at least an hour, all tire wet cake was removed from the filter, placed onto trays, and dried in a large tray dryer under vacuum at 40° C. for 48 hours. At this point the residual water and methanol on the cake was only 0.5 wt %. A total of 14.5 kg of dry cake was removed from the tray dryer indicating that a high yield of 93.5% was obtained, especially when physical losses are considered. The volume mean particle size was 8.8 μm with 95% of the particles less than 20.3 μm by volume. The surface area was 1.7 m² as measured by BET nitrogen adsorption. These results were comparable to the laboratory material of Example 10 demonstrating scale up of the process.

FIG. 21 can be compared to FIG. 19. Tire crystals were of similar size and shape. Here the sonication power per unit volume was reduced from 100 W per liter in the laboratory to <1 Watts per liter yet the performance was acceptable. Thus demonstrating that practical levels of sonication power can be used at all scales successfully.

### Example 11

### Compound D=Bisphosphonate

This example demonstrates scale up of a cooldown batch crystallization. It also demonstrates that for scale up, agglomeration of the crystals may be prevented by using a recycle loop with a turbulent flow rate (mean linear velocity of 1 m/s) and double tee energy device to help disperse the micro-seed aid product during crystallization. This example further demonstrates that it is possible to prevent agglomerates from forming without sonication.

Milling of Micro-Seed for Example 11

The procedure was similar to that of Example 10 except a DYNO®-Mill Type KDLA media mill was used with a different product feed stream. The DYNO®-Mill was charged with 495 ml 1.5 mm yttrium stabilized zirconium oxide beads, and deionized water was recycled through the mill to wet the heads. The excess water was then discarded. A total of 1.0 kg of Compound D was charged to 10 liters of deionized water in the 30 liter vessel. This charge corresponded to 3 wt % out of solution versus the main batch after accounting for the partial dissolution in the water. The slurry was recycled though the rotor/stator mill for 15 minutes and then aged overnight. The slurry was then recycled through the media mill via the Masterflex pump at a rate of 0.9 L/min. The mill tip speed was set at 6.8 m/s. The milling was conducted for 5 hours. The slurry was discharged from the mill into a drum. A sample of the slurry was filtered on a 0.2 um filter and washed with acetone (less than about 0.1 g/l solubility) to facilitate drying of the sample. The sample was dried in a vacuum oven and analyzed. The volume mean particle size was 3.19 um with 95% of the particles less than 7.8 um. The profile was uimodal. The surface area was 1.7 m²/g by nitrogen adsorption.

Crystallization for Example 11

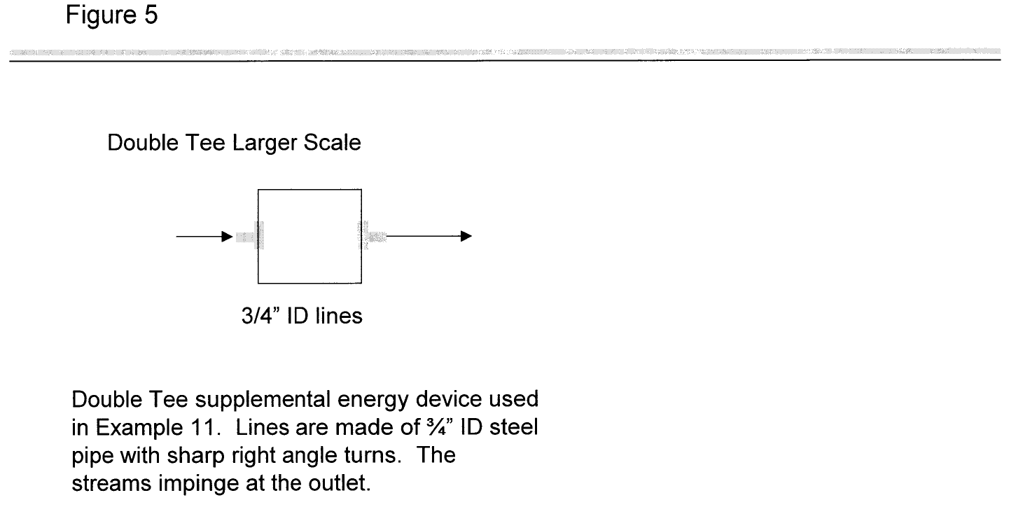

Mechanical setup: The same equipment setup for the crystallizer was used as for Example 10 above. The energy device consisted of a “Double Tee” as depicted in FIG. 5. The lines are made of ¾″ ID steel pipe with sharp right angle turns. The streams impinge at the outlet.

Batch Crystallization: A total of 22 kg of Compound D was charged to 220 liters of deionized water and dissolved at 60° C. The dissolved solution in the 100 gallon tank was agitated, maintained at 60° C. and recycled around the recycle loop at a flow rate of 29 kg/min. The batch was cooled to 51 to 52× to create supersaturation for the seed charge. The mean linear velocity (volumetric flow rate/cross sectional area) in the recycle line was 1.4 to 1.7 m/s for the majority of the line, and the turnover time of the batch was 9 minutes. In this example, the recycle line contained a double tee as the energy device along with a turbulent recycle loop. The vessel was agitated With a at 4 m/s tip speed.

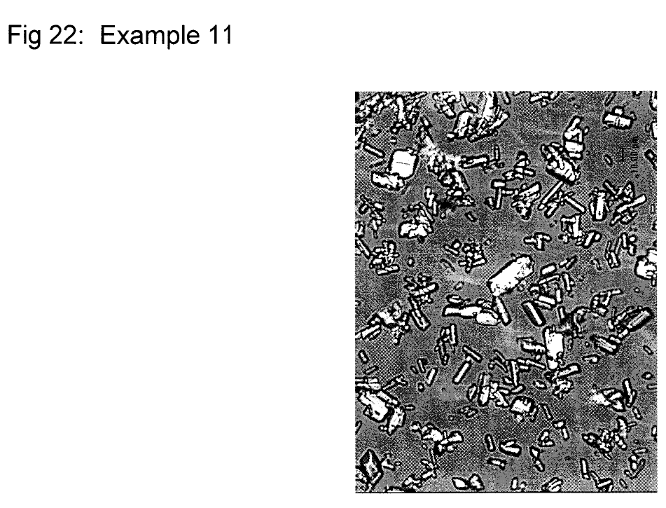

The micro-seed slurry was charged to the recycle loop via a diaphragm pump and ⅜″ seed charge port at a constant rate over 4 minutes. The charge was made directly into the recycle loop to facilitate dispersion of the seed slurry. The batch was cooled by the seed charge to 50 to 52° C., the batch was aged at this temperature for 30 minutes, and then cooled to 0.1 to 3° C. over 0.10 hours via controlled linear cooldown. An optical micrograph of the slurry was taken as shown in FIG. 22. As demonstrated in FIG. 22, the particles were monodispersed with no small particles due to uncontrolled nucleation present.

Post Processing of Example 11

Filtration and drying: After cooldown the batch was aged at 1 to 3° C. overnight, then filtered in a preceded (1 to 3° C.) agitated filter drier (Cogeim 0.25 m²) set with a poly filter cloth (KAVON™ brand 909 weave available from Shaffer, Inc.). The wet cake was washed with three consecutive 65 kg acetone slurry washes (consisting of the solvent charge, agitation of the contents for several minutes, and then filtration). These washes were utilized to remove the residual mother liquors of a product concentration high enough to lead to agglomeration of the solids during drying. The acetone washed solids were dried in the same filter under full vacuum with 25° C. fluid on the filter jacket and packaged. Micrographs indicated that there was no agglomeration of the cake, and the dry cake mean volume particle size was 20.6 μm. 95% of the particles were less than 41 mm by volume using the Helos dry particle analyzer. The surface area was 0.40 m²/g by BET nitrogen adsorption. These results are comparable to the lab scale experiments of Example 8B and C. This is in contrast to the results of Example 8A where insufficient particle dispersion was utilized during the crystallization.

### Example 12

### Compound D=Bisphosphonate

This example demonstrates flexibility in selection of operating conditions and choice of energy device for MMC on a given product. It is also the third example of production scale operations. This example used the same mechanical setup and procedure as Example 11, but was stressed by shortening the cooldown time from 10 hr to 3 hrs, and by increasing the turnover time from 9 minutes to 18 minutes. These actions result in more potential for nucleation and less frequent exposure to the recycle loop and energy device to break any agglomerates formed in the crystallizer into dispersed particles. The faster solids deposition rate and slower recycle rate through the energy device were offset by replacing the double tee with a higher intensity energy device, a Telsonic radial probe 12″ long and 2″ wide operated at an output of 800 W power in a 1 L flow through cell. The seed load was also increased to 10 wt % to obtain a significantly smaller product than Example 11.

Seed generation: The procedure followed that of Example 11 for the product and mill preparation. Here 3.48 kg of Compound D pure and 33 kg deionized water was charge to the to 30 L vessel and recycled around DYNO®-Mill Type KOLA at 0.45-0.9 L/min flow rate for 16 hours. The resultant particle size of the product was a mean volume of 2.8 μm and 95% of the particles less than 6.4 um. The surface area was 2.0 m²/g.

Batch Crystallization: The procedure matched that of Example 11 except that the 22 kg of Compound D dissolved in water in the 100 gallon tank was recycled around the recycle loop at a flow rate near 15 kg/min throughout the hatch. The batch was cooled to approximately 53-54° C. to create supersaturation for the seed charge.

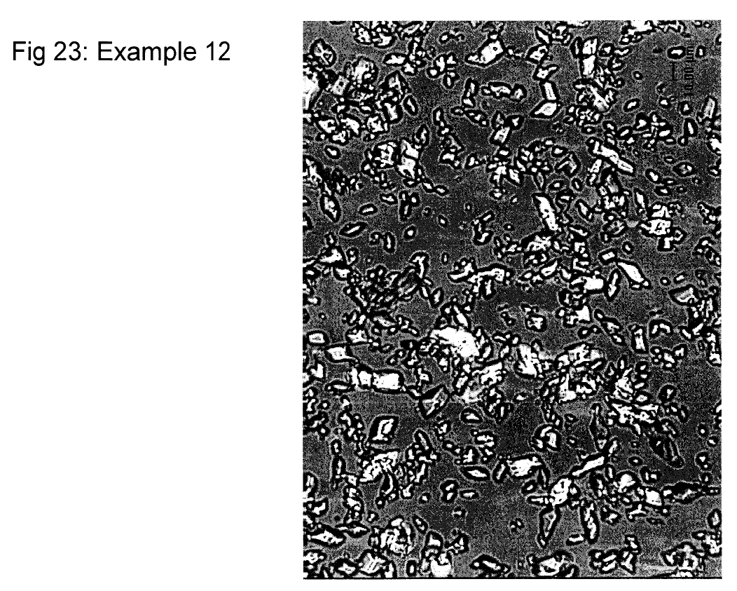

The micro-seed slurry was charged to the recycle loop via a diaphragm pump and ⅜″ seed charge port at a constant rate over 8 minutes. The charge was made directly into the recycle loop to facilitate dispersion of the seed slurry. The batch was cooled by the seed charge to about 50-52° C., the batch was aged at this temperature for 30 minutes, and then cooled to approximately 1-3° C. over 3 hours via controlled linear cooldown. An optical micrograph of the slurry was taken as in FIG. 23. FIG. 23 demonstrates that, the particles were monodispersed with no small particles due to uncontrolled nucleation present. The material was post processed by filtration, washed and dried as in Example 11. The crystallization conditions and results are shown below:

| | Example 12 | Example 11 |
|---|---|---|
| Batch Volume | 260 L | 240 L |
| Agitator Tip speed | 4 | 4 |
| (m/s) | | |
| Seed (%) | 10 | 3 |
| Seed charge time | 8 | 4 |
| (min.) | | |
| Cooldown Time | 3 | 10 |
| (hr) | | |
| Turnover Time | 18 | 9 |
| (min) | | |
| Energy Device | Sonicator (800 W) | Double Tee |
| Acetone washed | 3 × slurry | 3 × slurry |
| mv (um) | 11.6 | 20.61 |
| 95% < (um) | 23.8 | 40.34 |
| Surface area | 0.5686 | 0.4019 |
| sqm/g | | |
| Agglomerated | No | No |

The present application claims priority benefit of U.S. Provisional Patent Application Ser. No. 60/782,169 filed Mar. 14, 2006, which is hereby incorporated by reference in its entirety.

What is claimed:

1. A process for the production of crystalline particles of an organic active compound comprising subjecting micro-seed to a crystallization process, wherein the micro-seed is generated by a wet milling process and has a mean particle size of about 0.1 to about 20 μm and wherein the resulting crystalline particles have a mean particle size less than 100 μm.

2. The process of claim 1, wherein the mean particle size of the resulting crystalline particles is less than 60 μm.

3. The process of claim 1, wherein the mean size of the micro-seed is approximately 0.5 to 20 μm.

4. The process of claim 1, wherein the mean size of the micro-seed is approximately 1 to 10 μm.

5. The process of claim 1, wherein a cavitation mill, a ball mill, a media mill, or sonication is utilized during the wet milling process.

6. The process of claim 5, wherein the media mill or ball media, utilizes 0.5 to 4 mm beads.

7. The process of claim 6, wherein a ceramic mill and ceramic beads are utilized or a chromium-lined mill and ceramic beads are utilized.

8. The process of claim 1, wherein the organic active compound is a pharmaceutical.

9. The process of claim 8, wherein the pharmaceutical is selected from the group consisting of analgesics, anti-inflammatory agents, antihelmintics, anti-arrthymics, anti-asthmatics, antibiotics, anticoagulants, antidepressants, antidiabetic agents, anti epileptics, antihistamines, antihypertensive agents, antimuscarinic agents, antimycobacterial agents, antineoplastic agents, immunosuppressants, antithyroid agents, antiviral agents, anxiolytics, sedatives, astringents, beta-adrenergic receptor blocking drugs, contrast media, corticosteroids, cough suppressants, diagnostic agents, diagnostic imaging agents, dopaminergics, haemostatics, immunological agents, lipid regulating agents, muscle relaxants, parasympathomimetics, parathyroid calcitonin, prostaglandins, radio-pharmaceuticals, sex hormones, anti-allergic agents, stimulants, sympathomimetics, thyroid agents, vasodilators and xanthines.

10. A pharmaceutical composition comprising the crystalline particles produced in the process of claim 1 and a pharmaceutically acceptable carrier.

11. The process of claim 1, wherein the crystallization process comprises the following steps:
(1) generating a slurry of the micro-seed;
(2) generating a solution of the product to be crystallized; and
(3) combining the product of step (1) and the product of step (2).

12. The process of claim 11, wherein the crystallization process comprises using a batch, a semi-continuous or a continuous processing configuration.

13. The process of claim 12, wherein a recycle loop is utilized during the crystallization process.

14. The process of claim 11, wherein the solvent system of the crystallization process comprises primarily an aqueous solvent stream, primarily an organic solvent stream or a mixed solvent stream.

15. The process of claim 11, wherein a supplemental energy device is utilized during the cry stabilization process.

16. The process of claim 15, wherein the supplemental energy device is a mixing tee, a mixing elbow, a static mixer, a sonicator, or a rotor stator homogenizer.

17. The process of claim 15, wherein the supplemental energy device is utilized at the end of the crystallization process.

18. The process of claim 15, wherein the supplemental energy device is placed in a recycle loop.

19. The process of claim 11, wherein the crystallization process further comprises adding the micro-seed, a batch solution, a reagent solution, or an antisolvent into a recycle loop or a region of high mixing intensity.

20. The process of claim 11, wherein the crystallization process further comprises adding one or more supplemental additives.

21. The process of claim 11, wherein the slurry of the micro-seed and the solution of the product are rapidly micro-mixed when they are combined.

22. The process of claim 1, wherein the crystallization process comprises the following steps:
(1) generating a slurry of the micro seed using media milling;
(2) dissolving a portion of the micro-seed; and
(3) crystallizing the organic active compound on the micro-seed.

23. The process of claim L wherein the resulting crystalline particle have a crystalline form that corresponds to the form of the micro-seed.
